(19) United States
(12) Patent Application Publication
(10) Pub. No.: US 2021/0236012 A1
(43) Pub. Date: Aug. 5, 2021

(54) Blood pressure measurement device

(71) Applicants: OMRON Corporation, Kyoto (JP); OMRON HEALTHCARE Co., Ltd., Kyoto (JP)

(72) Inventors: Tomoyuki NISHIDA, Kyoto (JP); Takashi ONO, Kyoto (JP); Chisato TAWARA, Kyoto (JP); Yuichiro ARIMA, ARIMA (JP); Keitaro NAGANO, Kyoto (JP)

(21) Appl. No.: 17/224,427

(22) Filed: Apr. 7, 2021

Related U.S. Application Data
(63) Continuation of application No. PCT/JP2019/038388, filed on Sep. 27, 2019.

(30) Foreign Application Priority Data
Oct. 30, 2018 (JP) 2018-204204

(51) Int. Cl. A61B 5/022; A61B 5/021

(52) U.S. Cl. CPC A61B 5/02233; A61B 5/0235; A61B 5/02141

(57) ABSTRACT

A blood pressure measurement device enabling an increase in joining strength between a cuff and a junction target preventing an increase in size of the blood pressure measurement device. The device includes a case including an outer case having a tubular shape, a curler curving in such a manner as to follow along a circumferential direction of a portion of the wrist where the blood pressure measurement device is attached, the curler including a first facing portion aligned at one end of the outer case in a thickness direction, and a back-side cuff formed of two sheets formed of a resin material, the back-side cuff being configured to be inflated with a fluid, one of the sheet members that is disposed on the curler side including a second facing portion facing the first facing portion, and the second facing portion being larger than other portions of the back-side cuff.

### CROSS-REFERENCE TO RELATED APPLICATIONS

This application is the U.S. national stage application filed pursuant to 35 U.S.C. 365(c) and 120 as a continuation of International Patent Application No. PCT/JP2019/038388, filed Sep. 27, 2019, which application claims priority from Japanese Patent Application No. 2018-204204, filed Oct. 30, 2018, which applications are incorporated herein by reference in their entireties.

### TECHNICAL FIELD

The present invention relates to a blood pressure measurement device for measuring blood pressure.

### BACKGROUND ART

In recent years, blood pressure measurement devices for measuring blood pressure are being used to monitor health status at home, as well as in medical facilities. A blood pressure measurement device detects vibration of the artery wall to measure blood pressure by, for example, inflating and contracting a cuff wrapped around the upper arm or the wrist of a living body and detecting the pressure of the cuff using a pressure sensor.

As such a blood pressure measurement device, for example, a so-called integral type is known in which a cuff is integrated with a device body feeding a fluid to the cuff. Such blood pressure measurement devices pose a problem in that wrinkles, folds, or the like in the cuff reduce the accuracy of measurement results for the measured blood pressure. Additionally, in the blood pressure measurement device, the cuff needs to be inflated in the direction in which the blood vessels are occluded and to closely contact the wrist.

Thus, a technique for the blood pressure measurement device is known in which a curler is used between a belt and the cuff to bring the cuff inflated into close contact with the upper arm or the wrist (see, for example, JP 2018-102743 A). In such a blood pressure measurement device, the cuff is constituted to be integrated with the curler by bonding and fixing the cuff to the curler using, for example, a bonding layer such as double-sided tape.

### CITATION LIST

### Patent Literature

Patent Document 1: JP 2018-102743 A

### SUMMARY OF INVENTION

### Technical Problem

The blood pressure measurement device described above is required to improve the joining strength between the cuff and the curler. Thus, the joining strength may be improved by increasing a junction margin provided on the cuff or the curler.

However, for the above-described blood pressure measurement device, wearable devices attached to the wrist have recently been proposed. For this reason, it is not preferable that an increase in the junction margin provided on the cuff or the curler leads to an increased size of the blood pressure measurement device.

Thus, an object of the present invention is to provide a blood pressure measurement device that enables the joining strength between the cuff and the curler to be improved while preventing an increase in the size of the blood pressure measurement device.

### Solution to Problem

According to one aspect, a blood pressure measurement device is provided that includes, a case including an outer case having a tubular shape, a curler curving in such a manner as to follow along a circumferential direction of a portion of a living body where the blood pressure measurement device is attached, the curler including a first facing portion aligned at one end of the outer case in a thickness direction, and a cuff formed of two sheet members formed of a resin material, the cuff being configured to be inflated with a fluid, one of the sheet members that is disposed on the curler side including a second facing portion facing the first facing portion, and the second facing portion being larger than other portions of the cuff in a width direction.

Here, the fluid includes a liquid and air.

According to this aspect, the second facing portion of the cuff, which is large in the width direction, is joined to the curler to increase the junction margin, thus allowing the joining strength between the cuff and the curler to be improved. In the blood pressure measurement device according to the one aspect described above, the blood pressure measurement device is provided, in which the first facing portion is constituted in a shape being larger than other portions of the curler in a width direction.

According to this aspect, the first facing portion of the curler that increases a junction area corresponds to a region facing the end portion of the outer case in the thickness direction, thus allowing prevention of an increase in the size of the blood pressure measurement device due to an increase of the junction area. In other words, the first facing portion of the curler and the second facing portion of the cuff are aligned on the outer case in the thickness direction, the appearance of the blood pressure measurement device 1 is prevented from being significantly changed by the first facing portion and the second facing portion. In this way, an increase in the size of the blood pressure measurement device can be prevented.

In the blood pressure measurement device according to the one aspect described above, the blood pressure measurement device is provided, which includes a cover member sandwiching a portion of the second facing portion between the cover member and the first facing portion, the portion protruding beyond the other portions in the width direction.

According to this configuration, the cuff is joined to the curler by direct joining and to the curler with the cover member in between by indirect joining. Thus, the joining strength between the cuff and the curler can be increased.

In the blood pressure measurement device according to the one aspect described above, the blood pressure measurement device is provided, in which the cover member includes a protruding portion, the second facing portion includes a hole where a part of the protruding portion is disposed, and the first facing portion includes a fitting portion where a part of the protruding portion fits.

According to this aspect, the protruding portion of the cover member fits into the hole in the curler to increase the fixing strength between the cover member and the curler, thus increasing the joining strength between the cuff and the curler is improved.

In the blood pressure measurement device according to the one aspect described above, the blood pressure measurement device is provided, in which the case includes a back lid covering the one end of the outer case, and the first facing portion is connected to the back lid.

According to this aspect, the case can be assembled independently on the curler, thus increasing the degree of freedom in manufacturing the blood pressure measurement device.

In the blood pressure measurement device according to the one aspect described above, the blood pressure measurement device is provided, in which the first facing portion covers the one end of the outer case.

According to this aspect, the curler also serves as the back lid of the outer case, enabling a reduction in the number of components of the blood pressure measurement device.

### Advantageous Effects of Invention

The present invention can provide a blood pressure measurement device that enables the joining strength between the cuff and the curler.

### BRIEF DESCRIPTION OF DRAWINGS

FIG. 1 is a perspective view illustrating a configuration of a blood pressure measurement device according to a first embodiment of the present invention.

FIG. 2 is a perspective view illustrating the configuration of the blood pressure measurement device.

FIG. 3 is an exploded perspective view illustrating the configuration of the blood pressure measurement device.

FIG. 4 is an explanatory diagram illustrating a state in which the blood pressure measurement device is attached to the wrist.

FIG. 5 is a block diagram illustrating the configuration of the blood pressure measurement device.

FIG. 6 is a perspective view illustrating a configuration of a device body and a curler of the blood pressure measurement device.

FIG. 7 is a plan view illustrating a configuration of a cuff structure of the blood pressure measurement device.

FIG. 8 is a plan view illustrating another configuration of the cuff structure of the blood pressure measurement device.

FIG. 9 is a cross-sectional view illustrating a configuration of a belt, the curler, and the cuff structure of the blood pressure measurement device.

FIG. 10 is a cross-sectional view illustrating the configuration of the curler and the cuff structure of the blood pressure measurement device.

FIG. 11 is a plan view illustrating a configuration of an eighteenth sheet member of a back-side cuff of the blood pressure measurement device.

FIG. 12 is a cross-sectional view illustrating the configuration of the curler and the cuff structure of the blood pressure measurement device.

FIG. 13 is an explanatory diagram illustrating the configuration in which the cuff structure is inflated in a state in which the blood pressure measurement device is attached to the wrist.

FIG. 14 is a cross-sectional view illustrating the configuration in which the cuff structure is inflated in a state in which the blood pressure measurement device is attached to the wrist.

FIG. 15 is a flowchart illustrating an example of usage of the blood pressure measurement device.

FIG. 16 is a perspective view illustrating an example in which the blood pressure measurement device is attached to the wrist.

FIG. 17 is a perspective view illustrating an example in which the blood pressure measurement device is attached to the wrist.

FIG. 18 is a perspective view illustrating an example in which the blood pressure measurement device is attached to the wrist.

FIG. 19 is a perspective view illustrating a configuration of a blood pressure measurement device according to a second embodiment of the present invention.

FIG. 20 is a cross-sectional view illustrating the configuration of the blood pressure measurement device.

FIG. 21 is a bottom view illustrating a configuration of a main portion of a blood pressure measurement device according to a third embodiment.

FIG. 22 is a cross-sectional view illustrating a configuration of the main portion.

FIG. 23 is a perspective view illustrating a configuration of a main portion of a modified example of the blood pressure measurement device according to the third embodiment.

FIG. 24 is a bottom view of the configuration of the modified example of the blood pressure measurement device according to the third embodiment as viewed from the wrist side.

FIG. 25 is a cross-sectional view illustrating the configuration of the main portion.

FIG. 26 is a bottom view of the configuration of the main portion of the modified example of the blood pressure measurement device according to the third embodiment as viewed from the wrist side.

FIG. 27 is a cross-sectional view illustrating the configuration of the main portion.

FIG. 28 is a bottom view of the configuration of the main portion of the modified example of the blood pressure measurement device according to the third embodiment as viewed from the wrist side.

FIG. 29 is a cross-sectional view illustrating the configuration of the main portion.

### DESCRIPTION OF EMBODIMENTS

### First Embodiment

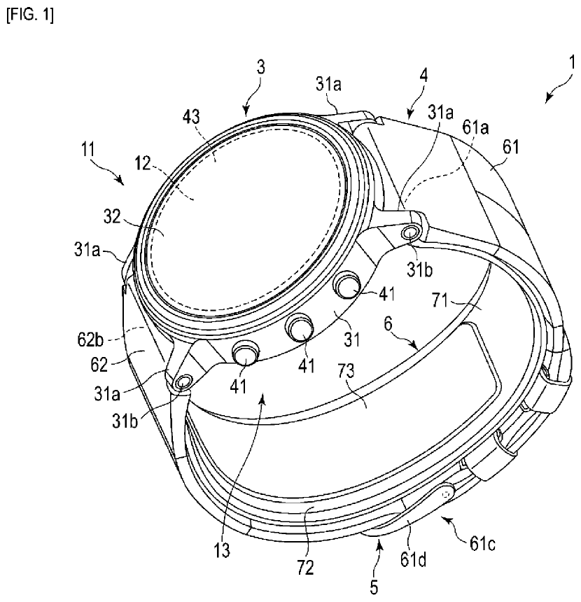

An example of a blood pressure measurement device 1 according to the first embodiment of the present invention is described below using FIGS. 1 to 18.

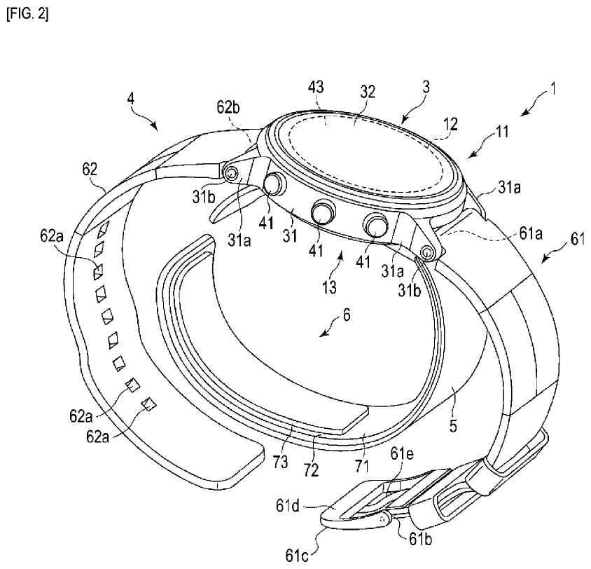

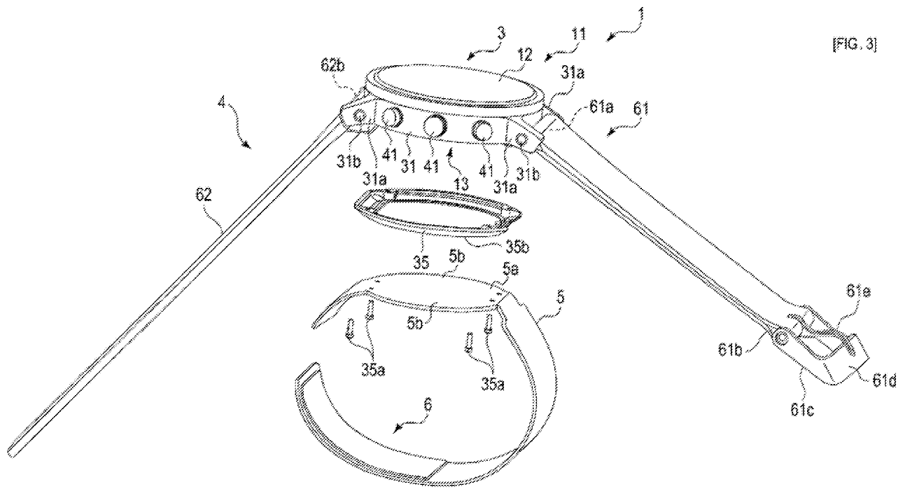

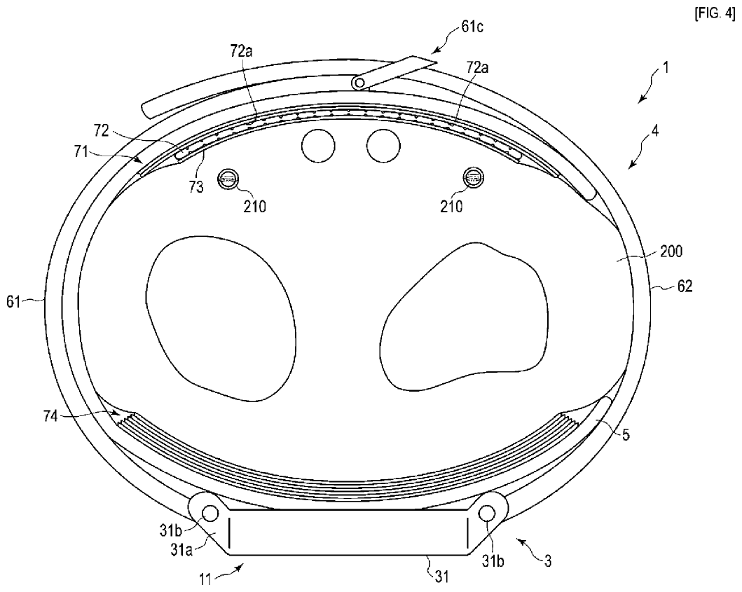

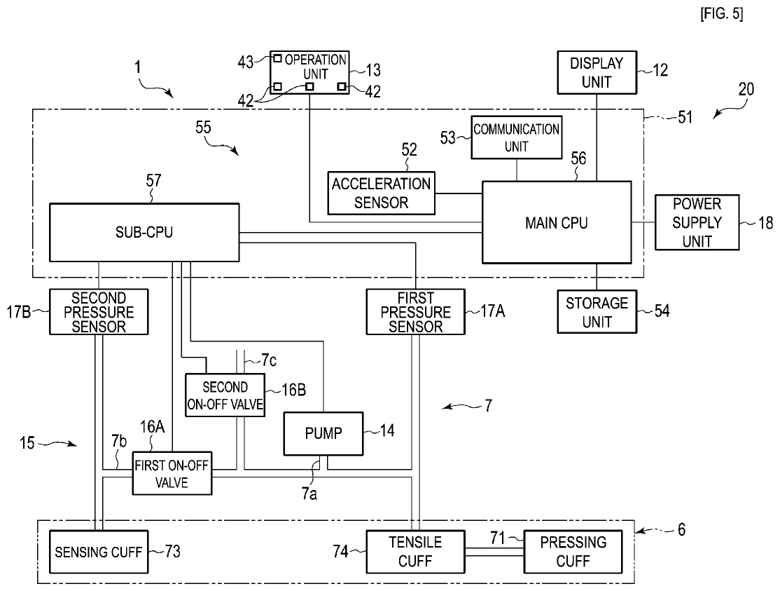

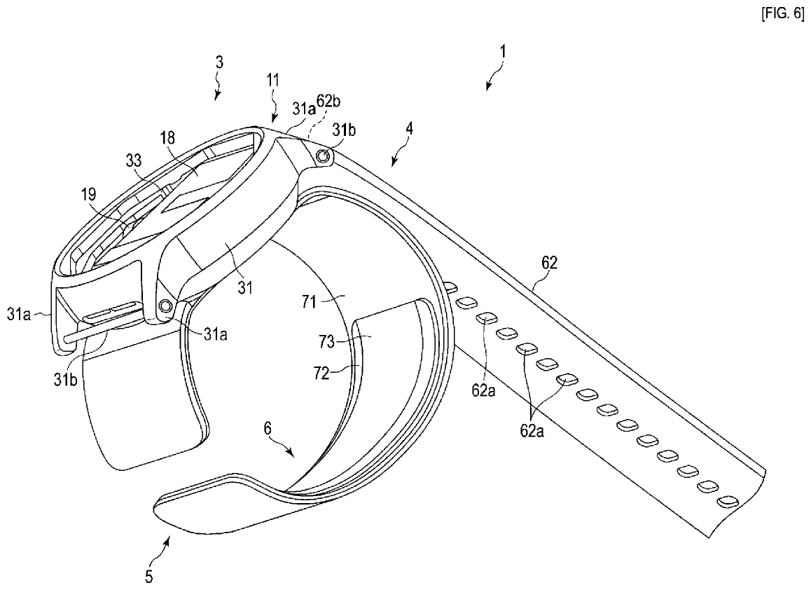

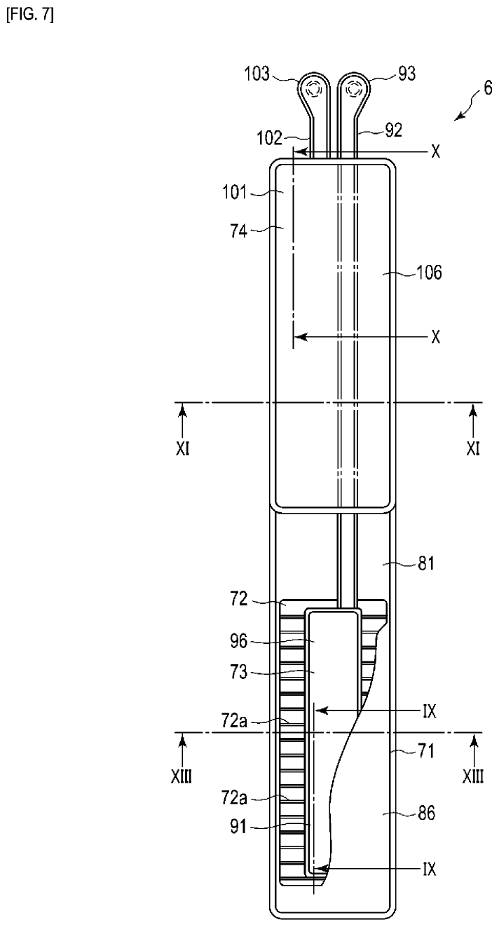

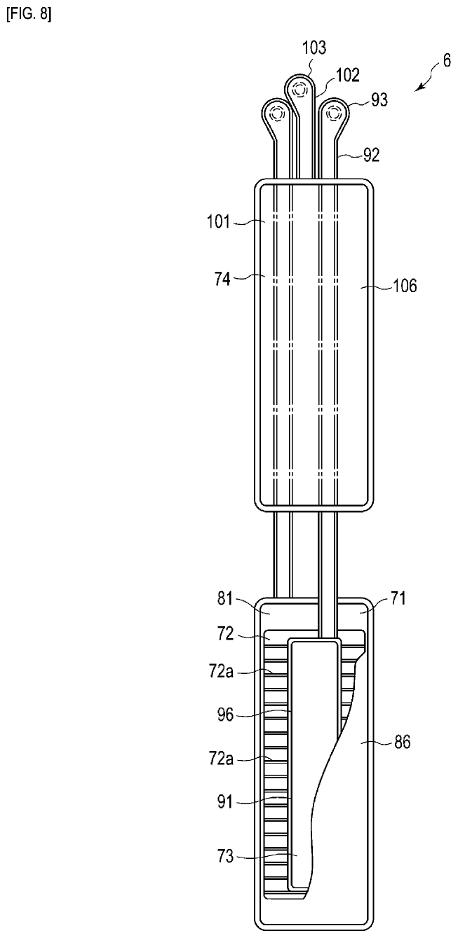

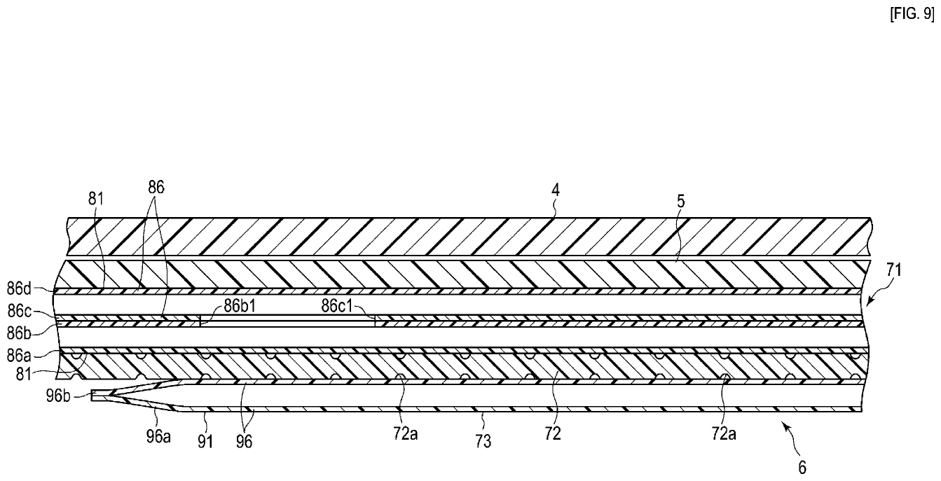

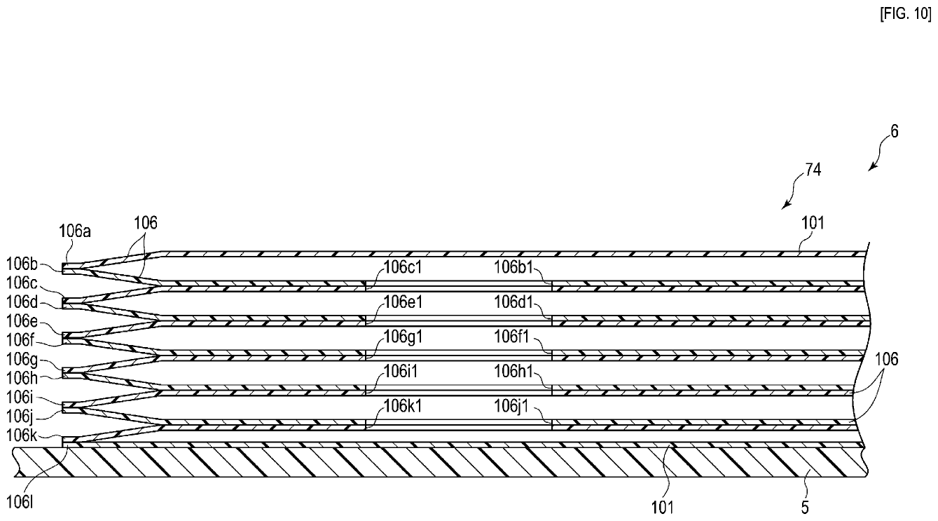

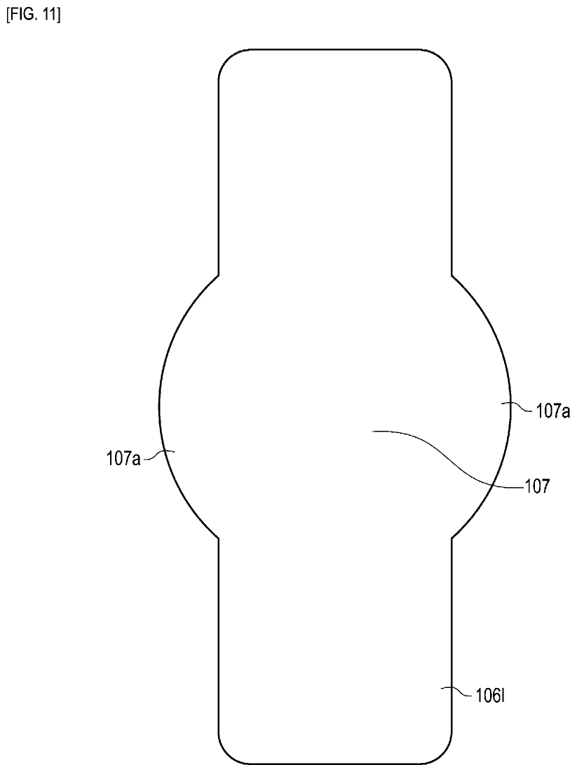

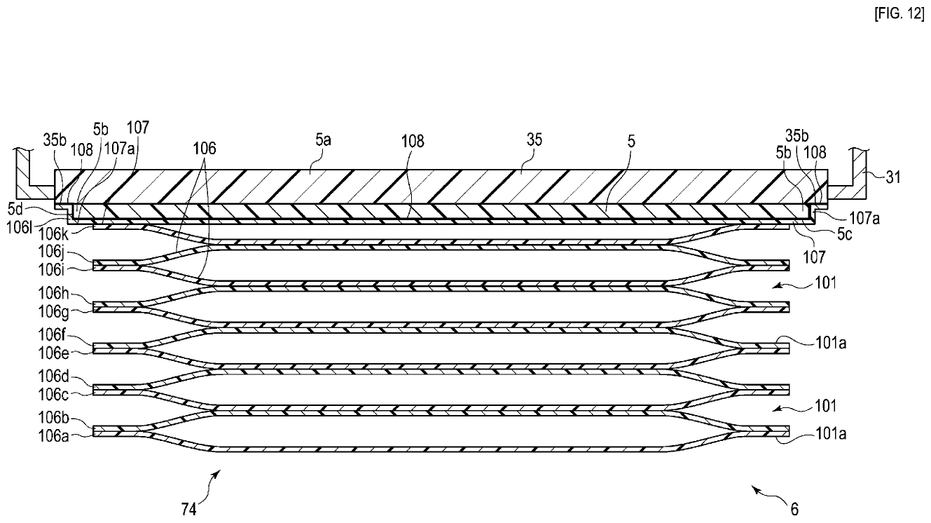

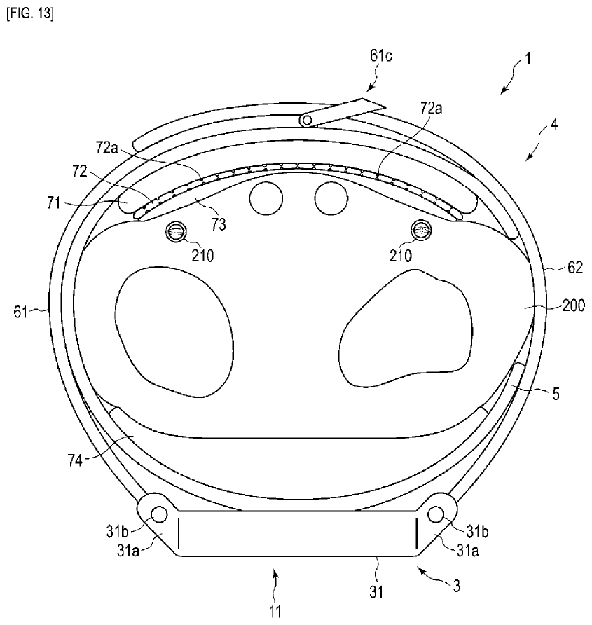

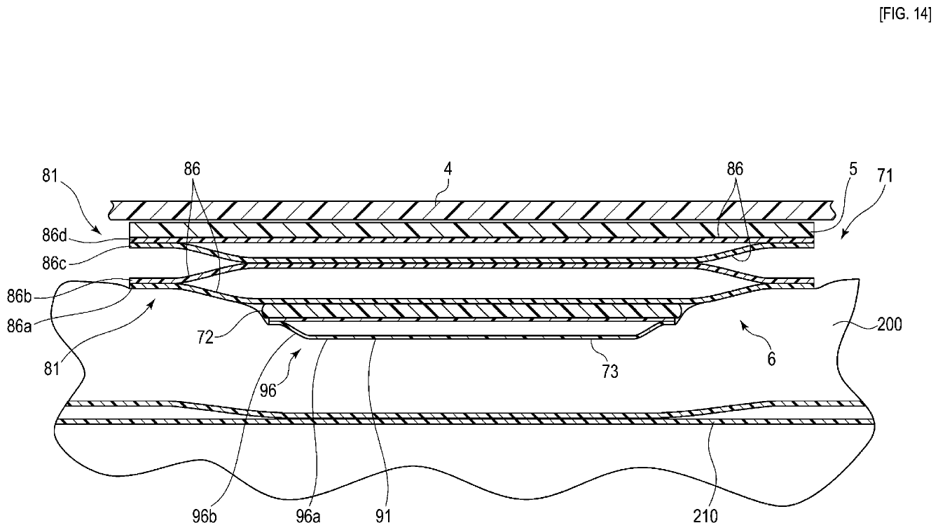

FIG. 1 is a perspective view illustrating a configuration of the blood pressure measurement device 1 according to an embodiment of the present invention in a state in which a belt 4 is closed. FIG. 2 is a perspective view illustrating the configuration of the blood pressure measurement device 1 in a state in which the belt 4 is open. FIG. 3 is an exploded perspective view illustrating the configuration of the blood pressure measurement device 1. FIG. 4 is an explanatory diagram illustrating, in cross section, a state in which the blood pressure measurement device 1 is attached to the wrist 200. FIG. 5 is a block diagram illustrating the configuration of the blood pressure measurement device 1. FIG. 6 is a perspective view illustrating a configuration of a device body 3 and a curler 5 of the blood pressure measurement device 1. FIG. 7 is a plan view illustrating a configuration of a cuff structure 6 of the blood pressure measurement device 1. FIG. 8 is a plan view illustrating another configuration of the cuff structure 6 of the blood pressure measurement device 1. FIG. 9 is a cross-sectional view illustrating a configuration of the belt 4, the curler 5, and the cuff structure 6 on a palm-side cuff 71 side of the blood pressure measurement device 1, which is taken along line IX-IX in FIG. 7. FIG. 10 is a cross-sectional view illustrating a configuration of the curler 5 and the cuff structure 6 on a back-side cuff 74 side of the blood pressure measurement device 1. FIG. 11 is a plan view illustrating a configuration of an eighteenth sheet member 106l of the back-side cuff 74 of the blood pressure measurement device 1. FIG. 12 is a cross-sectional view illustrating a configuration of the cuff structure 6 with the curler 5 and a tube 92 omitted, on the back-side cuff 74 side of the blood pressure measurement device 1, which is taken along line XI-XI in FIG. 7. FIG. 13 is an explanatory diagram illustrating the configuration in which the cuff structure 6 is inflated in a state in which the blood pressure measurement device 1 is attached to the wrist 200. FIG. 14 is a cross-sectional view illustrating the configuration in which the cuff structure 6 is inflated in a state in which the blood pressure measurement device 1 is attached to the wrist, which is taken along line XIV-XIV in FIG. 7.

The blood pressure measurement device 1 is an electronic blood pressure measurement device attached to a living body. The present embodiment will be described using an electronic blood pressure measurement device having an aspect of a wearable device attached to a wrist 200 of the living body.

As illustrated in FIGS. 1 to 3, the blood pressure measurement device 1 includes a device body 3, a belt 4 that fixes the device body 3 at the wrist, a curler 5 disposed between the belt 4 and the wrist, a cuff structure 6 including a palm-side cuff 71, a sensing cuff 73, and a back-side cuff 74, a fluid circuit 7 fluidly connecting the device body 3 and the cuff structure 6, and a joining member 8 joining the curler 5 and the cuff structure 6.

As illustrated in FIGS. 1 to 5, the device body 3 includes, for example, a case 11, a display unit 12, an operation unit 13, a pump 14, a flow path unit 15, an on-off valve 16, a pressure sensor 17, a power supply unit 18, a vibration motor 19, and a control substrate 20. The device body 3 feeds a fluid to the cuff structure 6 using the pump 14, the on-off valve 16, the pressure sensor 17, the control substrate 20, and the like.

As illustrated in FIGS. 1 to 3, the case 11 includes an outer case 31, a windshield 32 that covers an upper opening of the outer case 31, a base 33 provided at a lower portion of an interior of the outer case 31, and a back lid 35 covering a lower portion of the outer case 31.

The outer case 31 is formed in a cylindrical shape. The outer case 31 includes pairs of lugs 31a provided at respective symmetrical positions in the circumferential direction of an outer circumferential surface, and spring rods 31b each provided between the lugs 31 of each of the two pairs of lugs 31a. The windshield 32 is, for example, a circular glass plate.

The base portion 33 holds the display unit 12, the operation unit 13, the pump 14, the on-off valve 16, the pressure sensor 17, the power supply unit 18, the vibration motor 19, and the control substrate 20. Additionally, the base 33 constitutes a portion of the flow path unit 15 that makes the pump 14 and the cuff structure 6 fluidly continuous.

The back lid 35 covers a living body side end portion of the outer case 31. The back lid 35 is fixed to the living body side end portion of the outer case 31 or the base 33 using, for example, four screws 35a or the like. A surface shape of the back surface 35b of the back lid 35 is constituted in a circular shape because the outer case 31 is formed in a cylindrical shape. Moreover, the diameter of the back lid 35 is smaller than the diameter of the outer case 31. In other words, the length of the back lid 35 along the width direction of the curler 5 is shorter than the length of the outer case 31 along the width direction of the curler 5.

The display unit 12 is disposed on the base portion 33 of the outer case 31 and directly below the windshield 32. The display unit 12 is electrically connected to the control board 20. The display unit 12 is, for example, a liquid crystal display or an organic electroluminescence display. The display unit 12 displays various types of information including the date and time and measurement results of blood pressure values such as the systolic blood pressure and diastolic blood pressure, heart rate, and the like.

The operation unit 13 is configured to be capable of receiving an instruction input from a user. For example, the operation unit 13 includes a plurality of buttons 41 provided on the case 11, a sensor 42 that detects operation of the buttons 41, and a touch panel 43 provided on the display unit 12 or the windshield 32, as illustrated in FIG. 5. When operated by the user, the operation unit 13 converts an instruction into an electrical signal. The sensor 42 and the touch panel 43 are electrically connected to the control substrate 20 to output electrical signals to the control substrate 20.

As the plurality of buttons 41, for example, three buttons are provided. The buttons 41 are supported by the base 33 and protrude from the outer circumferential surface of the outer case 31. The plurality of buttons 41 and a plurality of the sensors 42 are supported by the base 33. The touch panel 43 is integrally provided on the windshield 32, for example.

The pump 14 is, for example, a piezoelectric pump. The pump 14 compresses air and feeds compressed air to the cuff structure 6 through the flow path unit 15. The pump 14 is electrically connected to the control substrate 20.

The flow path unit 15 constitutes a flow path connecting from the pump 14 to the palm-side cuff 71 and the back-side cuff 74 and a flow path connecting from the pump 14 to the sensing cuff 73, as illustrated in FIG. 5. Additionally, the flow path unit 15 constitutes a flow path connecting from the palm-side cuff 71 and the back-side cuff 74 to the atmosphere, and a flow path connecting from the sensing cuff 73 to the atmosphere. The flow path unit 15 is a flow path of air constituted by a hollow portion, a groove, a tube, or the like provided in the base portion 33 and the like.

The on-off valve 16 opens and closes a portion of the flow path 15. A plurality of the on-off valves 16 is provided, for example, as illustrated in FIG. 5, and selectively opens and closes the flow path connecting from the pump 14 to the palm-side cuff 71 and the back-side cuff 74, the flow path connecting from the pump 14 to the sensing cuff 73, the flow path connecting from the palm-side cuff 71 and the back-side cuff 74 to the atmosphere, and the flow path connecting from the sensing cuff 73 to the atmosphere, by the combination of opening and closing of each of the on-off valves 16. For example, two on-off valves 16 are used.

The pressure sensor 17 detects the pressures in the palm-side cuff 71, the sensing cuff 73 and the back-side cuff 74. The pressure sensor 17 is electrically connected to the control substrate 20. The pressure sensor 17 converts a detected pressure into an electrical signal, and outputs the electrical signal to the control substrate 20. The pressure sensor 17 is provided in the flow path connecting from the pump 14 to the palm-side cuff 71 and the back-side cuff 74 and in the flow path connecting from the pump 14 to the sensing cuff 73, as illustrated in FIG. 5. These flow paths are continuous through the palm-side cuff 71, the sensing cuff 73, and the back-side cuff 74, and thus the pressure in these flow paths corresponds to the pressure in the internal space of the palm-side cuff 71, the sensing cuff 73, and the back-side cuff 74.

The power supply unit 18 is, for example, a secondary battery such as a lithium ion battery. The power supply unit 18 is electrically connected to the control substrate 20. The power supply unit 18 supplies power to the control substrate 20.

As illustrated in FIGS. 5 and 6, the control substrate 20 includes, for example, a substrate 51, an acceleration sensor 52, a communication unit 53, a storage unit 54, and a control unit 55. The control substrate 20 is constituted by the acceleration sensor 52, the communication unit 53, the storage unit 54, and the control unit 55 that are mounted on the substrate 51.

The substrate 51 is fixed to the base 33 of the case 11 using screws or the like.

The acceleration sensor 52 is, for example, a 3-axis acceleration sensor. The acceleration sensor 52 outputs, to the control unit 55, an acceleration signal representing acceleration of the device body 3 in three directions orthogonal to one another. For example, the acceleration sensor 52 is used to measure, from the detected acceleration, the amount of activity of a living body to which the blood pressure measurement device 1 is attached.

The communication unit 53 is configured to be able to transmit and receive information to and from an external device wirelessly or by wire. For example, the communication unit 53 transmits information controlled by the control unit 55, and information of a measured blood pressure value, a pulse, and the like to an external device via a network, and receives a program or the like for software update from an external device via a network and sends the program or the like to the control unit 55.

In the present embodiment, the network is, for example, the Internet, but is not limited to this. The network may be a network such as a Local Area Network (LAN) provided in a hospital or may be direct communication with an external device using a cable or the like including a terminal of a predetermined standard such as a USB. Thus, the communication unit 53 may be configured to include a plurality of wireless antennas, micro-USB connectors, or the like.

The storage unit 54 pre-stores program data for controlling the overall blood pressure measurement device 1 and a fluid circuit 7, settings data for setting various functions of the blood pressure measurement device 1, calculation data for calculating a blood pressure value and a pulse from pressure measured by the pressure sensors 17, and the like. Additionally, the storage unit 54 stores information such as a measured blood pressure value and a measured pulse.

The control unit 55 is constituted by one or more CPUs, and controls operation of the overall blood pressure measurement device 1 and operation of the fluid circuit. The control unit 55 is electrically connected to and supplies power to the display unit 12, the operation unit 13, the pump 14, each of the on-off valves 16 and the pressure sensors 17. Additionally, the control unit 55 controls operation of the display unit 12, the pump 14, and the on-off valves 16, based on electrical signals output by the operation unit 13 and the pressure sensors 17.

For example, as illustrated in FIG. 5, the control unit 55 includes a main Central Processing Unit (CPU) 56 that controls operation of the overall blood pressure measurement device 1, and a sub-CPU 57 that controls operation of the fluid circuit 7. For example, the main CPU 56 obtains measurement results such as blood pressure values, for example, the systolic blood pressure and the diastolic blood pressure, and the heart rate, from electrical signals output by the pressure sensor 17, and outputs an image signal corresponding to the measurement results to the display unit 12.

For example, the sub-CPU 57 drives the pump 14 and the on-off valves 16 to feed compressed air to the palm-side cuff 71 and the sensing cuff 73 when an instruction to measure the blood pressure is input from the operation unit 13. In addition, the sub-CPU 57 controls driving and stopping of the pump 14 and opening and closing of the on-off valves 16 based on electrical signal output by the pressure sensors 17. The sub-CPU 57 controls the pump 14 and the on-off valves 16 to selectively feed compressed air to the palm-side cuff 71 and the sensing cuff 73 and selectively depressurize the palm-side cuff 71 and the sensing cuff 73.

As illustrated in FIGS. 1 to 3, the belt 4 includes a first belt 61 provided on a first pair of lugs 31a and a first spring rod 31b, and a second belt 62 provided on a second pair of lugs 31a and a second spring rod 31b. The belt 4 is wrapped around the wrist 200 with a curler 5 in between.

The first belt 61 is referred to as a so-called a parent and is configured like a band. The first belt 61 includes a first hole portion 61a provided at a first end portion of the first belt 61 and extending orthogonally to the longitudinal direction of the first belt 61, a second hole portion 61b provided at a second end portion of the first belt 61 and extending orthogonally to the longitudinal direction of the first belt 61, and a buckle 61c provided on the second hole portion 61b. The first hole portion 61a has an inner diameter at which the spring rod 31b can be inserted into the first hole portion 61a and at which the first belt 61 can rotate with respect to the spring rod 31b. In other words, the first belt 61 is rotatably held by the outer case 31 by disposing the first hole portion 61a between the pair of lugs 31a and around the spring rod 31b.

The second hole portion 61b is provided at a tip of the first belt 61. The buckle 61c includes a frame body 61d in a rectangular frame shape and a prong 61e rotatably attached to the frame body 61d. A side of the frame body 61d to which the prong 61e is attached is inserted into the second hole portion 61b, and the frame body 61d is mounted rotatably with respect to the first belt 61.

The second belt 62 is referred to as a so-called blade tip, and is configured in a band-like shape having a width at which the second belt 62 can be inserted into the frame body 61d. In addition, the second belt 62 includes a plurality of small holes 62a into which the prong 61e is inserted. Additionally, the second belt 62 includes a third hole portion 62b provided at first end portion of the second belt 62 and extending orthogonally to the longitudinal direction of the second belt 62. The third hole portion 62b has an inner diameter at which the spring rod 31b can be inserted into the third hole portion 62b and at which the second belt 62 can rotate with respect to the spring rod 31b. In other words, the second belt 62 is rotatably held by the outer case 31 by disposing the third hole portion 62b between the pair of lugs 31a and around the spring rod 31b.

Thus, the second belt 62 of the belt 4 as described above is inserted into the frame body 61d, and the prong 61e is inserted into the small hole 62a in the belt 4, the first belt 61 and the second belt 62 are integrally connected together, and the belt 4, together with the outer case 31, comes to have an annular shape following the wrist 200 along the circumferential direction.

As illustrated in FIG. 4, the curler 5 is constituted in a band-like shape that curves along the circumferential direction of the wrist. The curler 5 is formed with a first end and a second end spaced apart from each other. For example, a first end-side outer surface of the curler 5 is fixed to the back lid 35 of the device body 3. The first end and the second end of the curler 5 are disposed at positions where the first end and the second end protrude from the back lid 35. Furthermore, the first end and the second end of the curler 5 are located adjacent to each other at a predetermined distance from each other.

Additionally, the curler 5 is fixed to the back lid 35 such that the first end and the second end are located on one lateral side of the wrist 200 when the blood pressure measurement device 1 is attached to the wrist 200.

As a specific example, as illustrated in FIG. 3, the curler 5 includes a first facing portion 5a. The first facing portion 5a faces the back lid 35. In other words, the first facing portion 5a is aligned with the end portion of the outer case in the axial direction of the outer case 31.

The first facing portion 5a is fixed to the back lid 35 using screws 35a or the like. Note that as an example, the curler 5 is fixed to the living body-side end portion of the outer case 31 or the base 33 along with the back lid 35 using the screws 35a or the like. Note that the configuration has been described in which the back lid 35 is fixed to the living body-side end portion of the outer case 31 or the base 33 along with the curler 5 using the screws 35a, but no such limitation is intended. The curler 5, in other examples, may be fixed to the back surface 35b of the back lid 35 using a bonding layer including an adhesive, double-sided tape, or the like. Note that the back surface 35b is the wrist 200-side surface.

The first facing portion 5a is constituted in a shape that is wider compared to all portions of the curler 5 other than the first facing portion 5a. As an example, a surface shape of the first facing portion 5a is constituted in a smaller surface shape than that of the back surface 35b of the back lid 35. In other words, the surface shape of the first facing portion 5a is constituted in a smaller circular shape than that of the back surface 35b, which is constituted in a circular shape.

As a specific example, the first facing portion 5a includes first wing portions 5b respectively located on both sides of the curler 5 in the width direction, the first wing portions 5b protruding in the width direction compared to both side portions of the first facing portion 5a in the longitudinal direction of the curler 5. Each of the first wing portions 5b is constituted in a shape that is formed like an arc shape with an edge protruding outward in the width direction. As described above, the first facing portion 5a includes two first wing portions 5b and is thus constituted in a shape being larger in the width direction compared to both side portions of the first facing portion 5a in the longitudinal direction of the curler 5.

As a specific example, as illustrated in FIG. 1, FIG. 2, and FIG. 4, the curler 5 has a shape that curves along a direction orthogonal to the circumferential, in other words, along the circumferential direction of the wrist 200 in a side view from the longitudinal direction of the wrist 200. The curler 5 extends, for example, from the device body 3 through the hand back side of the wrist 200 and one lateral side of the wrist 200 to the hand palm side of the wrist 200 and toward the other lateral side of the wrist 200. Specifically, by curving along the circumferential direction of the wrist 200, the curler 5 is disposed across the most of the wrist 200 in the circumferential direction, with both ends of the curler 5 spaced at a predetermined distance from each other.

The curler 5 has hardness appropriate to provide flexibility and shape retainability. Here, “flexibility” refers to deformation of the shape of the curler 5 in a radial direction at the time of application of an external force of the belt 4 to the curler 5. For example, “flexibility” refers to deformation of the shape of the curler 5 in a side view in which the curler 5 approaches the wrist, is along the shape of the wrist, or follows to the shape of the wrist when the curler 5 is pressed by the belt 4. Furthermore, “shape retainability” refers to the ability of the curler 5 to maintain a pre-imparted shape when no external force is applied to the curler 5. For example, “shape retainability” refers to, in the present embodiment, the ability of the curler 5 to maintain a shape that curves along the circumferential direction of the wrist.

The cuff structure 6 is disposed on an inner circumferential surface of the curler 5, and is held along the shape of the inner circumferential surface of the curler 5. As a specific example, the palm-side cuff 71 and the back-side cuff 74 are disposed on the inner circumferential surface of the curler 5, and the palm-side cuff 71 and the back-side cuff 74 are joined using the joining member 8.

The curler 5 is formed of a resin material. The curler 5 is formed of, for example, a thermoplastic resin material, and specifically, polypropylene. The curler 5 is formed, for example, to a thickness of approximately 1 mm.

As illustrated in FIGS. 1 to 4 and 7 to 14, the cuff structure 6 includes the palm-side cuff (cuff) 71, a back plate 72, the sensing cuff 73, and the back-side cuff (cuff) 74. The cuff structure 6 is fixed to the curler 5. The cuff structure 6 includes the palm-side cuff 71, the back plate 72, and the sensing cuff 73 that are stacked one another and disposed on the curler 5, and the back-side cuff 74 that is spaced apart from the palm-side cuff 71, the back plate 72, and the sensing cuff 73 and disposed on the curler 5.

As a specific example, the cuff structure 6 includes the palm-side cuff 71, the back plate 72, the sensing cuff 73, and the back-side cuff 74 that are disposed on an inner surface of the curler 5. The cuff structure 6 is fixed to the inner surface of the curler 5 on the hand palm side of the wrist 200 with the palm-side cuff 71, the back plate 72, and the sensing cuff 73 stacked in this order from the inner surface of the curler 5 toward the living body. In addition, the cuff structure 6 includes the back-side cuff 74 disposed on the inner surface of the curler 5 on the hand back side of the wrist 200. Each of the members of the cuff structure 6 is fixed to an adjacent member of the cuff structure 6 in a stacking direction with a double-sided tape, an adhesive, or the like.

The palm-side cuff 71 is a so-called pressing cuff. The palm-side cuff 71 is fluidly connected to the pump 14 through the flow path unit 15. The palm-side cuff 71 is inflated to press the back plate 72 and the sensing cuff 73 toward the living body side. The palm-side cuff 71 includes a plurality of, for example, two-layer air bags 81, and a plurality of insertion holes 82 formed in one of the two-layer air bags 81 disposed on the curler 5 side.

Here, the air bags 81 are bag-like structures, and in the present embodiment, the blood pressure measurement device 1 is configured to use air with the pump 14, and thus the present embodiment will be described using the air bags. However, in a case where a fluid other than air is used, the bag-like structures may be fluid bags such as liquid bags. The plurality of air bags 81 are stacked and are in fluid communication with one another in the stacking direction.

Each of the air bags 81 is constituted in a rectangular shape that is long in one direction. The air bag 81 is constituted, for example, by combining two sheet members 86 that are long in one direction, and thermally welding edges of the sheet members. In other words, the air bag 81 includes welded portions 81a formed by welding edge portions of four sides of the air bag 81.

As a specific example, as illustrated in FIGS. 7 to 9, the two-layer air bags 81 include a first sheet member 86a, a second sheet member 86b, a third sheet member 86c, and a fourth sheet member 86d in this order from the living body side. The second sheet member 86b constitutes a first-layer air bag 81 along with the first sheet member 86a, the third sheet member 86c is integrally bonded to the second sheet member 86b, and the fourth sheet member 86d constitutes a second-layer air bag 81 along with the third sheet member 86c. Note that the two-layer air bags 81 are integrally constituted by joining each of the sheet members 86 of the adjacent air bags 81 by bonding with a double-sided tape, an adhesive, or the like, or welding or the like.

Edge portions of four sides of the first sheet member 86a are welded to corresponding edge portions of four sides of the second sheet member 86b to constitute the air bag 81. The second sheet member 86b and the third sheet member 86c are disposed facing each other, and each includes a plurality of openings 86b1 and 86c1 through which the two air bags 81 are fluidly continuous.

Edge portions of four sides of the third sheet member 86c are welded to corresponding edge portions of four sides of the fourth sheet member 86d to constitute the air bag 81.

The back plate 72 is applied to an outer surface of the first sheet member 86a of the palm-side cuff 71 with an adhesive layer, a double-sided tape, or the like. The back plate 72 is formed in a plate shape using a resin material. The back plate 72 is made of polypropylene, for example, and is formed into a plate shape having a thickness of approximately 1 mm. The back plate 72 has shape followability.

Here, “shape followability” refers to a function of the backplate 72 by which the back plate 72 can be deformed in such a manner as to follow the shape of a contacted portion of the wrist 200 to be disposed, the contacted portion of the wrist 200 refers to a region of the wrist 200 that is faced by the back plate 72, and the contact as used herein includes both direct contact and indirect contact with the sensing cuff 73 in between.

For example, as illustrated in FIG. 9, the back plate 72 includes a plurality of grooves 72a formed in both main surfaces of the back plate 72 and extending in a direction orthogonal to the longitudinal direction. As illustrated in FIG. 9, a plurality of the grooves 72a are provided in both main surfaces of the back plate 72. The plurality of grooves 72a provided in one of the main surfaces face the corresponding grooves 72a provided in the other main surface in the thickness direction of the back plate 72. Additionally, the plurality of grooves 72a are disposed at equal intervals in the longitudinal direction of the back plate 72.

In the back plate 72, portions including the plurality of grooves 72a are thinner than portions including no grooves 72a and thus the portions including the plurality of grooves 72a are easily deformed. Accordingly, the back plate 72 is deformed in such a manner as to follow to the shape of the wrist 200, and has shape followability of extending in the circumferential direction of the wrist. The back plate 72 is formed such that the length of the back plate 72 is sufficient to cover the hand palm side of the wrist 200. The back plate 72 transfers the pressing force from the palm-side cuff 71 to the back plate 72 side main surface of the sensing cuff 73 in a state in which the back plate 72 is extending along the shape of the wrist 200.

The sensing cuff 73 is fixed to the living body side main surface of the back plate 72. The sensing cuff 73 is in direct contact with a region of the wrist 200 where an artery 210 resides, as illustrated in FIGS. 9 and 14. The artery 210 as used herein is the radial artery and the ulnar artery. The sensing cuff 73 is formed in the same shape as that of the back plate 72 or a shape that is smaller than that of the back plate 72, in the longitudinal direction and the width direction of the back plate 72. The sensing cuff 73 is inflated to compress a hand palm-side region of the wrist 200 in which the artery 210 resides. The sensing cuff 73 is pressed by the inflated palm-side cuff 71 toward the living body side with the back plate 72 in between.

As a specific example, the sensing cuff 73 includes one air bag 91, a tube 92 that communicates with the air bag 91, and a connection portion 93 provided at a tip of the tube 92. One main surface of the air bag 91 of the sensing cuff 73 is fixed to the back plate 72. For example, the sensing cuff 73 is applied to the living body side main surface of the back plate 72 using a double-sided tape, an adhesive layer, or the like.

Here, the air bag 91 is a bag-like structure, and in the present embodiment, the blood pressure measurement device 1 is configured to use air with the pump 14, and thus the present embodiment will be described using the air bag. However, in a case where a fluid other than air is used, the bag-like structure may be a liquid bag and the like.

The air bag 91 is constituted in a rectangular shape that is long in one direction. The air bag 91 is constituted, for example, by combining two sheet members 96 that are long in one direction, and thermally welding edges of the sheet members. As a specific example, the air bag 91 includes a fifth sheet member 96a and a sixth sheet member 96b in this order from the living body side as illustrated in FIG. 14.

For example, the fifth sheet member 96a and the sixth sheet member 96b are fixed by welding, with a tube 92 that is fluidly continuous with the internal space of the air bag 91 being disposed on one side of each of the fifth sheet member 96a and the sixth sheet member 96b. For example, the fifth sheet member 96a and the sixth sheet member 96b are welded together integrally with the tube 92 by welding edge portions of four sides of the fifth sheet member 96a to corresponding edge portions of four sides of the sixth sheet member 96b in a state in which the tube 92 is disposed between the fifth sheet member 96a and the sixth sheet member 96b.

The tube 92 is provided at one longitudinal end portion of the air bag 91. As a specific example, the tube 92 is provided at an end portion of the air bag 91 near the device body 3. The tube 92 includes the connection portion 93 at the tip. The tube 92 is connected to the flow path unit 15 and constitutes a flow path between the device body 3 and the air bag 91. The connection portion 93 is connected to the flow path unit 15. The connection portion 93 is, for example, a nipple.

The back-side cuff 74 is a so-called tensile cuff. The back-side cuff 74 is fluidly connected to the pump 14 through the flow path unit 15. The back-side cuff 74 is inflated to press the curler 5 such that the curler 5 is spaced apart from the wrist 200, pulling the belt 4 and the curler 5 toward the hand back side of the wrist 200. The back-side cuff 74 includes air bags 101 including a plurality of, for example, six layers, a tube 102 in communication with the air bags 101, and a connection portion 103 provided at a tip of the tube 102.

Additionally, the back-side cuff 74 is configured such that the thickness of the back-side cuff 74 in an inflating direction, in the present embodiment, in the direction in which the curler 5 and the wrist 200 face each other, during inflation, is larger than the thickness of the palm-side cuff 71 in the inflating direction during inflation and than the thickness of the sensing cuff 73 in the inflating direction during inflation. Specifically, the air bags 101 of the back-side cuff 74 include more layers than the air bags 81 in the palm-side cuff 71 and the air bag 91 in the sensing cuff 73, and are thicker than the palm-side cuff 71 and the sensing cuff 73 when the air bags 101 are inflated from the curler 5 toward the wrist 200.

Here, the air bag 101 is a bag-like structure, and in the present embodiment, the blood pressure measurement device 1 is configured to use air with the pump 14, and thus the present embodiment will be described using the air bag. However, in a case where a fluid other than air is used, the bag-like structure may be a fluid bag such as a liquid bag. A plurality of the air bags 101 are stacked and are in fluid communication in the stacking direction.

The air bag 101 is constituted in a rectangular shape that is long in one direction. The air bag 101 is constituted, for example, by combining two sheet members 106 that are long in one direction, and thermally welding edges of the sheet members. In other words, the air bag 101 includes welded portions 101a formed by welding edge portions of four sides of the air bag 101.

As a specific example, as illustrated in FIG. 10, the six-layer air bags 101 include a seventh sheet member 106a, an eighth sheet member 106b, a ninth sheet member 106c, a tenth sheet member 106d, an eleventh sheet member 106e, a twelfth sheet member 106f, a thirteenth sheet member 106g, a fourteenth sheet member 106h, a fifteenth sheet member 106i, a sixteenth sheet member 106j, a seventeenth sheet member 106k, and an eighteenth sheet member 106l in this order from the living body side. Note that the six-layer air bags 101 are integrally constituted by joining each of the sheet members 106 of the adjacent air bags 101 by bonding with a double-sided tape, an adhesive, or the like, or welding or the like.

Edge portions of four sides of the seventh sheet member 106a are welded to corresponding edge portions of four sides of the eighth sheet member 106b to constitute a first-layer air bag 101. The eighth sheet member 106b and the ninth sheet member 106c are disposed facing each other and are integrally bonded together. The eighth sheet member 106b and the ninth sheet member 106c include a plurality of openings 106b1 and 106c1 through which the adjacent air bags 101 are fluidly continuous. Edge portions of four sides of the ninth sheet member 106c are welded to corresponding edge portions of four sides of the tenth sheet member 106d to constitute a second-layer air bag 101.

The tenth sheet member 106d and the eleventh sheet member 106e are disposed facing each other and are integrally bonded together. The tenth sheet member 106d and the eleventh sheet member 106e include a plurality of openings 106d1 and 106e1 through which the adjacent air bags 101 are fluidly continuous. Edge portions of four sides of the eleventh sheet member 106e are welded to corresponding edge portions of four sides of the twelfth sheet member 106f to constitute a third-layer air bag 101.

The twelfth sheet member 106f and the thirteenth sheet member 106g are disposed facing each other and are integrally bonded together. The twelfth sheet member 106f and the thirteenth sheet member 106g include a plurality of openings 106f1 and 106g1 through which the adjacent air bags 101 are fluidly continuous. Edge portions of four sides of the thirteenth sheet member 106g are welded to corresponding edge portions of four sides of the fourteenth sheet member 106h to constitute a fourth-layer air bag 101.

The fourteenth sheet member 106h and the fifteenth sheet member 106i are disposed facing each other and are integrally bonded together. The fourteenth sheet member 106h and the fifteenth sheet member 106i include a plurality of openings 106h1 and 106i1 through which the adjacent air bags 101 are fluidly continuous. Edge portions of four sides of the fifteenth sheet member 106i are welded to corresponding edge portions of four sides of the sixteenth sheet member 106j to constitute a fifth-layer air bag 101.

The sixteenth sheet member 106j and the seventeenth sheet member 106k are disposed facing each other and are integrally bonded together. The sixteenth sheet member 106j and the seventeenth sheet member 106k include a plurality of openings 106j1 and 106k1 through which the adjacent air bags 101 are fluidly continuous. Edge portions of four sides of the seventeenth sheet member 106k are welded to corresponding edge portions of four sides of the eighteenth sheet member 106l to constitute a sixth-layer air bag 101. The eighteenth sheet member 106l is disposed on the curler 5 side.

In addition, for example, a tube 102 that is fluidly continuous with the internal space of the air bag 101 is disposed on one side of the seventeenth sheet member 106k and the eighteenth sheet member 106l, and is fixed by welding. For example, in a state in which the tube 102 is disposed between the seventeenth sheet member 106k and the eighteenth sheet member 106l, the edge portions of the seventeenth sheet member 106k are welded to the edge portions of the eighteenth sheet member 106l in a rectangular frame shape to form the air bag 101. Thus, the tube 102 is integrally welded to the air bag 101.

For example, the sixth-layer air bag 101 as described above is constituted integrally with the second layer air bag 81 of the palm-side cuff 71. Specifically, the seventeenth sheet member 106k is constituted integrally with the third sheet member 86c, and the eighteenth sheet member 106l is constituted integrally with the fourth sheet member 86d.

In more detail, the third sheet member 86c and the seventeenth sheet member 106k constitute a rectangular sheet member that is long in one direction, and the eighteenth sheet member 106l and the fourth sheet member 86d constitute a rectangular sheet member that is long in one direction. Then, these sheet members are stacked one another, and welding is performed such that first end portion side is welded in a rectangular frame shape, whereas a part of one side on the second end portion side is not welded. Thus, the second-layer air bag 81 of the palm-side cuff 71 is constituted. Then, welding is performed such that the second end portion side is welded in a rectangular frame shape, whereas a part of one side on the first end portion side is not welded. Thus, the sixth-layer air bag 101 in the back-side cuff 74 is constituted. In addition, a part of one side on the facing side of each of the second-layer air bag 81 and the sixth-layer air bag 101 is not welded, and thus the second-layer air bag 81 and the sixth-layer air bag 101 are fluidly continuous.

As illustrated in FIG. 11 and FIG. 12, the eighteenth sheet member 106l includes a second facing portion 107 that is joined to the first facing portion 5a of the curler 5.

The second facing portion 107 is a portion of the eighteenth sheet member 106l facing the first facing portion 5a, and is constituted in a shape being larger in the width direction compared to portions of the eighteenth sheet member 106l not facing the first facing portion 5a.

Furthermore, a surface shape of the second facing portion 107 is constituted in a larger surface shape than that of the first facing portion 5a. As a specific example, the second facing portion 107 includes second wing portions 107a extending in the width direction compared to both side portions of the second facing portion 107 in the longitudinal direction of the eighteenth sheet member 106l.

One second wing portion 107a is formed on each of the both sides in the width direction of the eighteenth sheet member 106l. As an example, the second wing portion 107a is constituted in a shape with an arc-like edge having a larger diameter than the arc-like edge of the first wing portion 5b of the first facing portion 5a.

The second facing portion 107 is joined to the first facing portion 5a by being bonded by a bonding layer 108 constituted by an adhesive, a double-sided tape, or the like. A surface shape of the second wing portion 107a is constituted in a larger surface shape than that of the first wing portion 5b, and thus the second wing portions 107a are joined to the back surface 5c of the first wing portion 5b, the side surface 5d of the first wing portion 5b, and the back surface 35b of the back lid 35 by bonding with the bonding layer 108. The region of the second facing portion 107 except for the second wing portions 107a is joined to the region of the first facing portion 5a except for the first wing portions 5b by bonding with the bonding layer 108.

Note that the configuration has been described in which a part of the second wing portion 107a is joined to the back surface 35b of the back lid 35, but no such limitation is intended. In other examples, the second wing portions 107a may be configured to be joined exclusively to the first facing portion 5a.

The tube 102 is connected to one air bag 101 of the six-layer air bags 101 and is provided at one longitudinal end portion of the air bag 101. As a specific example, the tube 102 is provided on the curler 5 side of the six-layer air bags 101 and is provided at the end portion close to the device body 3. The tube 102 includes a connection portion 103 at the tip. The tube 102 constitutes a flow path included in the fluid circuit 7 and located between the device body 3 and the air bags 101. The connection portion 103 is, for example, a nipple.

Note that, as described above, in the present embodiment, the configuration has been described in which a part of the back-side cuff 74 is constituted integrally with the palm-side cuff 71 and is fluidly continuous with the palm-side cuff 71. However, no such limitation is intended. For example, as illustrated in FIG. 8, the back-side cuff 74 may be constituted separately from the palm-side cuff 71 and be fluidly discontinuous with the palm-side cuff 71. For such a configuration, the palm-side cuff 71 may be configured such that, like the sensing cuff 73 and the back-side cuff 74, the palm-side cuff 71 is further provided with a tube and a connection portion, and in the fluid circuit 7 as well, the palm-side cuff 71 is connected to a flow path through which the fluid is fed to the palm-side cuff 71, a check valve, and a pressure sensor.

Additionally, each of the sheet members 86, 96, and 106 forming the palm-side cuff 71, the sensing cuff 73, and the back-side cuff 74 are formed of a thermoplastic resin material. The thermoplastic resin material is a thermoplastic elastomer. Examples of thermoplastic resin material constituting the sheet members 86, 96, and 106 include thermoplastic polyurethane based resin (hereinafter referred to as TPU), polyvinyl chloride resin, ethylene-vinyl acetate resin, thermoplastic polystyrene based resin, thermoplastic polyolefin resin, thermoplastic polyester based resin, and thermoplastic polyamide resin.

For example, the sheet members 86, 96, and 106 are formed using a molding method such as T-die extrusion molding or injection molding. After being molded by each molding method, the sheet members 86, 96, and 106 are sized into predetermined shapes, and the sized individual pieces are joined by welding or the like to constitute bag-like structures 81, 91, and 101. A high frequency welder or laser welding is used as the welding method.

The fluid circuit 7 is constituted by the case 11, the pump 14, the flow path unit 15, the on-off valves 16, the pressure sensors 17, the palm-side cuff 71, the sensing cuff 73, and the back-side cuff 74. A specific example of the fluid circuit 7 will be described below with two on-off valves 16 that are used in the fluid circuit 7 being designated as a first on-off valve 16A and a second on-off valve 16B, and two pressure sensors 17 that are used in the fluid circuit 17 being designated as a first pressure sensor 17A and a second pressure sensor 17B.

As illustrated in FIG. 5, the fluid circuit 7 includes, for example, a first flow path 7a that makes the palm-side cuff 71 and the back-side cuff 74 continuous with the pump 14, a second flow path 7b constituted by branching from a middle portion of the first flow path 7a and making the sensing cuff 73 continuous with the pump 14, and a third flow path 7c connecting the first flow path 7a to the atmosphere. Additionally, the first flow path 7a includes the first pressure sensor 17A. The first on-off valve 16A is provided between the first flow path 7a and the second flow path 7b. The second flow path 7b includes a second pressure sensor 17B. The second on-off valve 16B is provided between the first flow path 7a and the third flow path 7c.

In the fluid circuit 7 as described above, the first on-off valve 16A and the second on-off valve 16B are closed to connect only the first flow path 7a to the pump 14, and the pump 14 and the palm-side cuff 71 are fluidly connected. In the fluid circuit 7, the first on-off valve 16A is opened and the second on-off valve 16B is closed to connect the first flow path 7a and the second flow path 7b, thus fluidly connecting the pump 14 and the back-side cuff 74, the back-side cuff 74 and the palm-side cuff 71, and the pump 14 and the sensing cuff 73. In the fluid circuit 7, the first on-off valve 16A is closed and the second on-off valve 16B is opened to connect the first flow path 7a and the third flow path 7c, fluidly connecting the palm-side cuff 71, the back-side cuff 74, and the atmosphere together. In the fluid circuit 7, the first on-off valve 16A and the second on-off valve 16B are opened to connect the first flow path 7a, the second flow path 7b, and the third flow path 7c, fluidly connecting the palm-side cuff 71, the sensing cuff 73, the back-side cuff 74, and the atmosphere together.

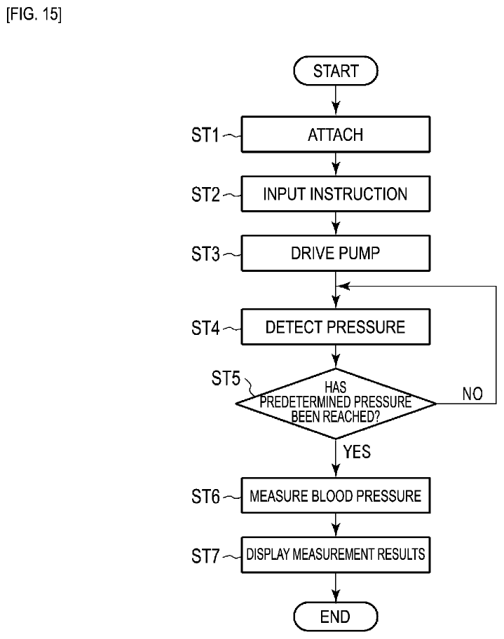

Now, an example of measurement of a blood pressure value using the blood pressure measurement device 1 will be described using FIGS. 15 to 18. FIG. 15 is a flowchart illustrating an example of a blood pressure measurement using the blood pressure measurement device 1, illustrating both an operation of a user and an operation of the control unit 55. Additionally, FIGS. 16 to 18 illustrate an example in which the blood pressure measurement device 1 is attached to the wrist 200 of the user.

First, the user attaches the blood pressure measurement device 1 to the wrist 200 (step ST1). As a specific example, for example, a user inserts one of the wrists 200 into the curler 5, as illustrated in FIG. 26.

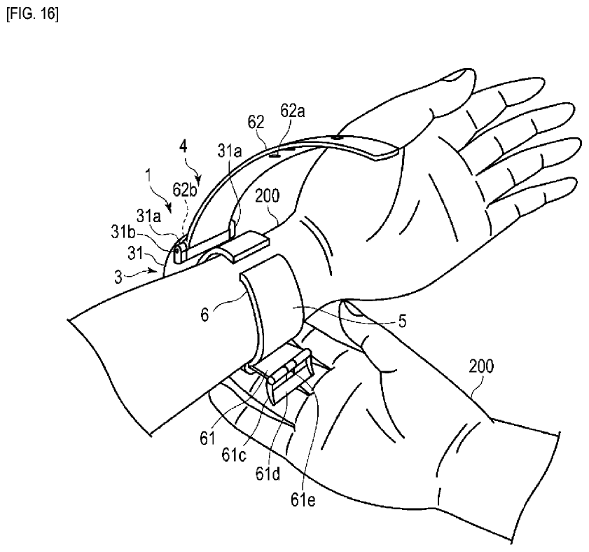

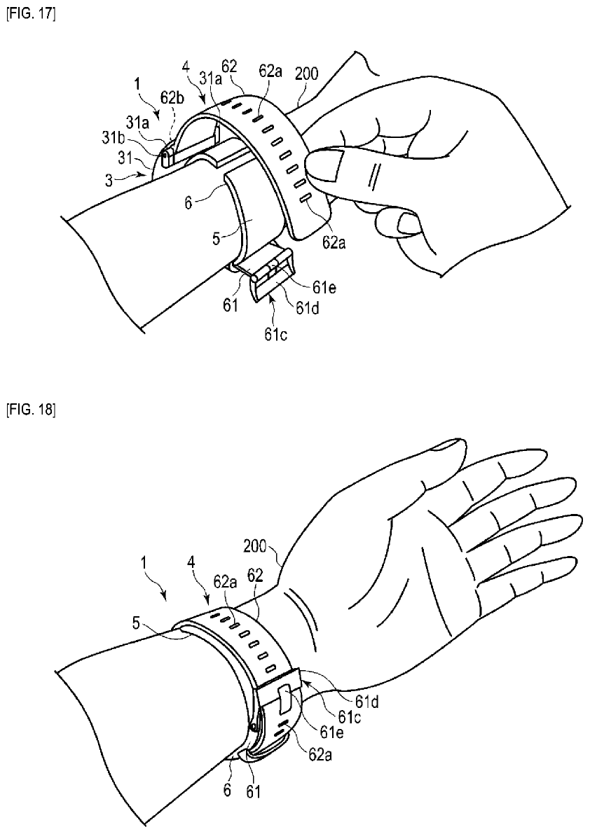

At this time, in the blood pressure measurement device 1, the device body 3 and the sensing cuff 73 are disposed at opposite positions in the curler 5, and thus the sensing cuff 73 is disposed in a region on the hand palm side of the wrist 200 in which the artery 210 resides. Thus, the device body 3 and the back-side cuff 74 are disposed on the hand back side of the wrist 200. Then, as illustrated in FIG. 17, the user passes the second belt 62 through the frame body 61d of the buckle 61c of the first belt 61 with the hand opposite to the hand on which the blood pressure measurement device 1 is disposed. The user then pulls the second belt 62 to bring the member on the inner circumferential surface side of the curler 5, that is, the cuff structure 6, into close contact with the wrist 200, and inserts the prong 61e into the small hole 62a. Thus, as illustrated in FIG. 18, the first belt 61 and the second belt 62 are connected, and the blood pressure measurement device 1 is attached to the wrist 200.

Then, the user operates the operation unit 13 to input an instruction corresponding to the start of measurement of the blood pressure value. The operation unit 13, on which an input operation of the instruction has been performed, outputs an electrical signal corresponding to the start of the measurement to the control unit 55 (step ST2). The control unit 55 receives the electrical signal, and then for example, opens the first on-off valve 16A, closes the second on-off valve 16B, and drives the pump 14 to feed compressed air to the palm-side cuff 71, the sensing cuff 73, and the back-side cuff 74 through the first flow path 7a and the second flow path 7b (step ST3). Thus, the palm-side cuff 71, the sensing cuff 73, and the back-side cuff 74 start to be inflated.

The first pressure sensor 17A and the second pressure sensor 17B detect the pressures in the palm-side cuff 71, the sensing cuff 73, and the back-side cuff 74, and outputs, to the control unit 55, electrical signals corresponding to the pressures (step ST4). Based on the received electrical signals, the control unit 55 determines whether the pressures in the internal spaces of the palm-side cuff 71, the sensing cuff 73, and the back-side cuff 74 have reached a predetermined pressure for measurement of the blood pressure (step ST5). For example, in a case where the internal pressures of the palm-side cuff 71 and the back-side cuff 74 have not reached the predetermined pressure and the internal pressure of the sensing cuff 73 has reached the predetermined pressure, the control unit 55 closes the first on-off valve 16A and feeds compressed air through the first flow path 7a.

When the internal pressures of the palm-side cuff 71 and the back-side cuff 74 and the internal pressure of the sensing cuff 73 all have reached the predetermined pressure, the control unit 55 stops driving the pump 14 (YES in step ST5). At this time, as illustrated in FIGS. 13 and 14, the palm-side cuff 71 and the back-side cuff 74 are sufficiently inflated, and the inflated palm-side cuff 71 presses the back plate 72. Additionally, the back-side cuff 74 presses against the curler 5 in a direction away from the wrist 200, and then the belt 4, the curler 5, and the device body 3 move in a direction away from the wrist 200, and as a result, the palm-side cuff 71, the back plate 72, the sensing cuff 73 and a flat plate 75 are pulled toward the wrist 200 side. In addition, when the belt 4, the curler 5, and the device body 3 move in a direction away from the wrist 200 due to the inflation of the back-side cuff 74, the belt 4 and the curler 5 move toward both lateral sides of the wrist 200, and the belt 4, the curler 5, and the device body 3 move in a state of close contact with both lateral sides of the wrist 200. Thus, the belt 4 and the curler 5, which are in close contact with the skin of the wrist 200, pull the skin on both lateral sides of the wrist 200 toward the hand back side.

Further, the sensing cuff 73 is inflated by being fed with a predetermined amount of air such that the internal pressure equals the pressure required to measure blood pressure, and is pressed toward the wrist 200 by the back plate 72 that is pressed by the palm-side cuff 71. Thus, the sensing cuff 73 presses the artery 210 in the wrist 200 and occludes the artery 210 as illustrated in FIG. 14.

Additionally, the control unit 55, for example, controls the second on-off valve 16B and repeats the opening and closing of the second on-off valve 16B, or adjusts the degree of opening of the second on-off valve 16B to pressurize the internal space of the palm-side cuff 71. In the process of pressurization, based on the electrical signal output by the second pressure sensor 17B, the control unit 55 obtains measurement results such as blood pressure values, for example, the systolic blood pressure and the diastolic blood pressure, and the heart rate and the like (step ST6). The control unit 55 outputs an image signal corresponding to the obtained measurement results to the display unit 12, and displays the measurement results on the display unit 12 (step ST7). In addition, after the end of the blood pressure measurement, the control unit 55 opens the first on-off valve 16A and the second on-off valve 16B.

The display unit 12 receives the image signal, and then displays the measurement results on the screen. The user views the display unit 12 to confirm the measurement results. After the measurement is complete, the user removes the prong 61e from the small hole 62a, removes the second belt 62 from the frame body 61 d, and removes the wrist 200 from the curler 5, thus removing the blood pressure measurement device 1 from the wrist 200.

In the blood pressure measurement device 1 according to the first embodiment configured as described above, the first facing portion 5a of the curler 5 includes the first wing portions 5b, and the second facing portion 107 of the eighteenth sheet member 106l disposed on the curler 5 side of the back-side cuff 74 includes the second wing portions 107a. The second wing portions 107a are joined to the first wing portions 5b by being bonded to the first wing portions 5b using the bonding layer 108. In this way, the junction area is increased in which the wing portions 5a1 and 107a join the back-side cuff 74 and the curler 5. Thus, the blood pressure measurement device 1 can increase the joining strength between the back-side cuff 74 and the curler 5. Furthermore, the use of the first wing portion portions 5b and the second wing portions 107 leads to a configuration in which only parts of the curler 5 and the back-side cuff 74 have an increased junction margin, allowing prevention of an increase in the size of the blood pressure measurement device.

In addition, the configuration is employed in which a surface shape of the second facing portion 107 is formed in a wider surface shape than that of the first facing portion 5a and in which a part of the second wing portion 107a is also fixed to the back lid 35, and thus the back-side cuff 74 can be joined directly to the case 11, thus enabling an increase in the strength at which the back-side cuff 74 is fixed to the case 11.

As described above, according to the blood pressure measurement device 1 according to the present embodiment, the joining strength between the back-side cuff 74 and the curler 5 can be increased.

### Second Embodiment

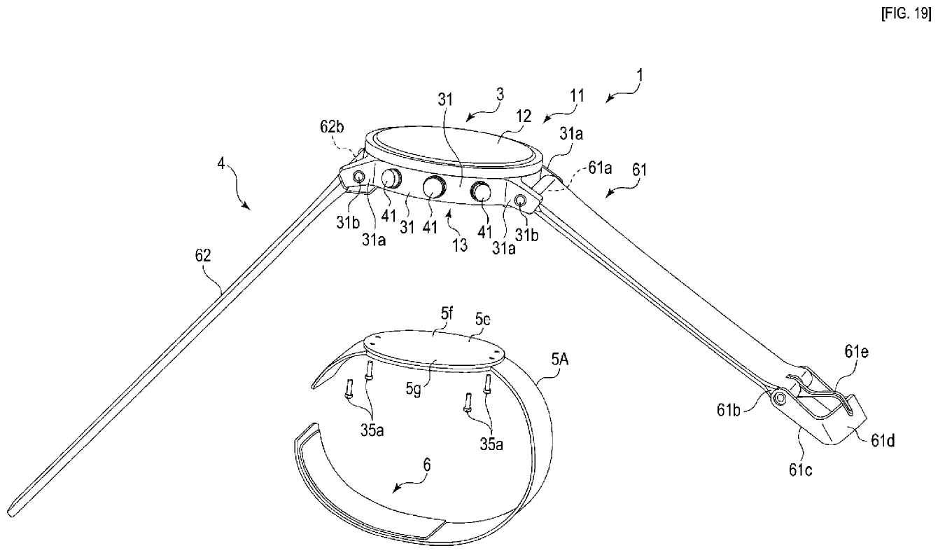

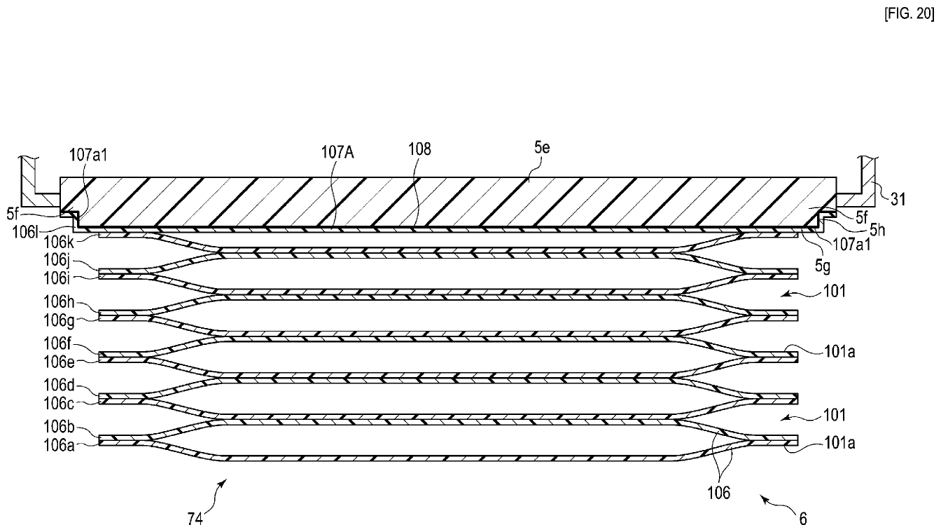

Now, a second embodiment of the blood pressure measurement device 1 will be described using FIGS. 19 and 20. Note that the blood pressure measurement device 1 according to the second embodiment is configured such that the curler 5 and the back lid 35 are integrally formed and that in this regard, the blood pressure measurement device 1 according to the second embodiment differs from the blood pressure measurement device 1 according to the first embodiment described above. Thus, components of the blood pressure measurement device 1 of the second embodiment that are similar to the corresponding components of the blood pressure measurement device 1 according to the first embodiment described above are denoted by the same reference signs in the description, and descriptions and illustrations of these components are omitted as appropriate. Note that in the second embodiment, a curler having a shape in which the curler 5 and the back lid 35 are integrally formed is referred to as a curler 5A.

FIG. 19 is an exploded perspective view illustrating a configuration of the blood pressure measurement device 1 according to the second embodiment. FIG. 20 is a cross-sectional view illustrating the configuration of the blood pressure measurement device 1 according to the second embodiment.

As illustrated in FIGS. 19 and 20, the case 11 includes an outer case 31, a windshield 32 that covers an upper opening of the outer case 31, and a base 33 provided at a lower portion of an interior of the outer case 31.

As illustrated in FIG. 19, the curler 5A is constituted in a band-like shape that curves along the circumferential direction of the wrist. The curler 5A is formed with a first end and a second end spaced apart from each other. The curler 5A includes a lid portion 5e that covers a living body-side end portion of the outer case 31.

The curler 5A is disposed at a position where one end and the other second end of the curler 5A protrude from the lid portion 5e. Furthermore, the first end and the second end of the curler 5A are located adjacent to each other at a predetermined distance from each other.

The lid portion 5e is fixed to the living body-side end portion of the outer case 31 or the base 33 using the screws 35a or the like. Additionally, the lid portion 5e is provided on the curler 5A such that when the blood pressure measurement device 1 is attached to the wrist 200, one end and the other end of the curler 5A are located on one lateral side of the wrist 200.

The lid portion 5e is constituted in a shape that is wider compared to all portions of the curler 5A other than the lid portion 5e. A surface shape of the lid portion 5e is constituted in a circular shape, as an example. As a specific example, the lid portion 5e is constituted in a shape including third wing portions 5f respectively located on both sides of the lid portion 5e in the width direction and protruding outward in the width direction compared to both side portions of the lid portions 5e in the longitudinal direction of the curler 5A. The lid portion 5e includes two third wing portions 5f, and are thus constituted to be larger compared to both side portions of the lid portion 5e in the longitudinal direction of the curler 5A. As an example, the third wing portions 5f are constituted in a shape having an arc-like edge protruding outward in the width direction.

As illustrated in FIG. 19 and FIG. 20, a cutout portion 5h is formed at the arc-like edge of the back surface 5g of a wrist 200 side of the third wing portion 5f of the lid portion 5e. The cutout portion 5h is formed in the back surface 5g, and thus the vicinity of the edge of the back surface 5g is constituted as a step.

The back surface 5g configured as described above is, for example, constituted in the same shape as that of a wrist 200 side surface of an integral body of the back lid 35f and the first facing portion 5s of the curler 5 of the blood pressure measurement device 1 according to the first embodiment. Note that the third wing portion 5f is not limited to the configuration in which the surface shape of the third wing portion 5f includes an arc-like shape edge. The third wing portion 5f may be constituted to have a rectangular surface shape, for example.

Additionally, the curler 5A has a shape that curves along a direction orthogonal to the circumferential direction of the wrist 200, in other words, along the circumferential direction of the wrist 200 in a side view from the longitudinal direction of the wrist 200. The curler 5A extends, for example, from the device body 3 through the hand back side of the wrist 200 and the one lateral side of the wrist 200 to the hand palm side of the wrist 200 and toward the other lateral side of the wrist 200. Specifically, by curving along the circumferential direction of the wrist 200, the curler 5A is disposed across the most of the wrist 200 in the circumferential direction, with both ends of the curler 5 spaced at a predetermined distance from each other.

The curler 5A has hardness appropriate to provide flexibility and shape retainability. Here, “flexibility” refers to deformation of the shape of the curler 5A in the radial direction at the time of application of an external force of the belt 4 to the curler 5A. For example, “flexibility” refers to deformation of the shape of the curler 5A in a side view in which the curler 5A approaches the wrist, is along the shape of the wrist, or follows to the shape of the wrist when the curler 5A is pressed by the belt 4. Furthermore, “shape retainability” refers to the ability of the curler 5A to maintain a pre-imparted shape when no external force is applied to the curler 5A. For example, “shape retainability” refers to, in the present embodiment, the ability of the curler 5A to maintain a shape that curves along the circumferential direction of the wrist.

The cuff structure 6 is disposed on an inner circumferential surface of the curler 5A, and is held along the shape of the inner circumferential surface of the curler 5A. As a specific example, the palm-side cuff 71 and the back-side cuff 74 are disposed on the inner circumferential surface of the curler 5A, and the palm-side cuff 71 and the back-side cuff 74 are joined using the joining member 8.

The curler 5A is formed of a resin material. The curler 5A is formed of, for example, a thermoplastic resin material, and specifically, polypropylene. The curler 5A is formed, for example, to a thickness of approximately 1 mm.

The curler 5A configured in this manner has, for example, a configuration in which the back lid 35 and the facing portion 5a of the blood pressure measurement device 1 according to the first embodiment are integrally formed.

The eighteenth sheet member 106l includes a second facing portion 107A joined to the lid portion 5e of the curler 5A. The second facing portion 107A is a portion of the eighteenth sheet member 106l that faces the lid portion 5e, and is constituted in a shape being larger in the width direction compared to a portion of the eighteenth sheet member 106l not facing the lid portion 5e.

Furthermore, a surface shape of the second facing portion 107A is constituted in a larger surface shape than that of the lid portion 5e, for example. As a specific example, the second facing portion 107A includes second wing portions 107a1 extending in the width direction compared to both side portions of the second facing portion 107A in the longitudinal direction of the eighteenth sheet member 106l. One second wing portion 107a1 is formed on each of the both sides in the width direction of the eighteenth sheet member 106l. As an example, the second wing portions 107a1 in a surface shape is constituted in a shape including an arc-like edge having a larger diameter than the arc-like edge of the third wing portions 5f of the lid portion 5e, as an example.

The second facing portion 107A is joined to the lid portion 5e by being bonded using the bonding layer 108 constituted by an adhesive, a double-sided tape, or the like. Because the surface shape of the second facing portion 107A is constituted in a larger surface shape than that of the first facing portion 5a, the second wing portions 107a1 is bonded to the third wing portions 5f of the lid portion 5e by being bonded using the bonding layer 108. In addition, as an example, the second wing portions 107a1 is also joined to the cutout portion 5h of the third wing portion 5f. The region of the second facing portion 107A a excluding the second wing portions 107a1 is joined by bonding by the bonding layer 108 to a region of the lid portion 5e excluding the third wing portion 5f.

Note that a configuration in which a portion of the second wing portions 107a1 is joined to the notch portion 5h of the lid portion 5e has been described, but no such limitation is intended. In other examples, the second wing portions 107a1 may have a configuration in which the second wing portions 107a1 has a shape that is joined to a region other than the cutout portion 5h, of the back surface 5g of the third wing portion 5f on the wrist 200 side.

The blood pressure measurement device 1 according to the second embodiment configured as described above produces effects similar to the effects of the first embodiment. Furthermore, the number of components constituting the blood pressure measurement device 1 can be reduced.

### Third Embodiment

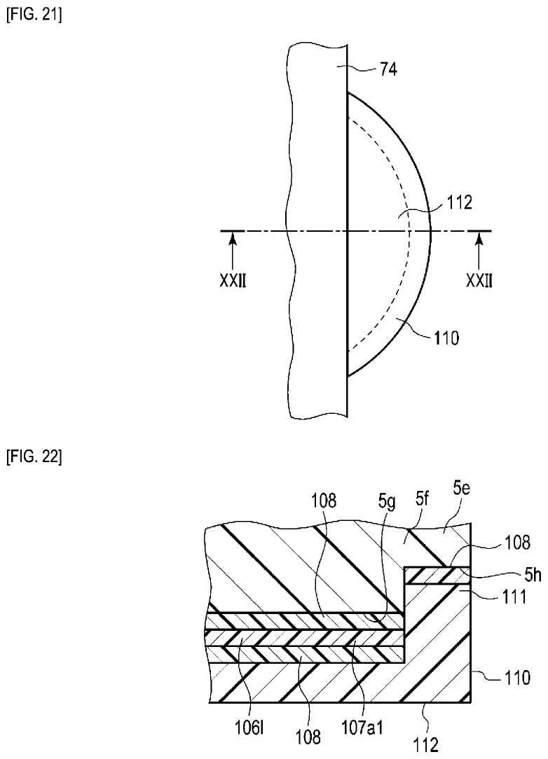

Now, a third embodiment of the blood pressure measurement device 1 will be described using FIGS. 21 and 22. Note that the blood pressure measurement device 1 according to the third embodiment is configured to include a cover member 110 that sandwiches the second wing portions 107a1 of the eighteenth sheet member 106l of the back-side cuff 74 between the cover member 110 and the third wing portions 5f of the lid portion 5e of the curler 5A, and in this regard, differs from the blood pressure measurement device 1 according to the second embodiment described above.

Thus, components of the blood pressure measurement device 1 of the third embodiment that are similar to the corresponding components of the blood pressure measurement device 1 according to the first embodiment described above are denoted by the same reference signs in the description, and descriptions and illustrations of these components are omitted as appropriate.

FIG. 21 is a bottom view of the vicinity of one of the two second wing portions 107a1 of the back-side cuff 74 as viewed from the wrist 200 side. FIG. 22 is a cross-sectional view illustrating one of the two second wing portions 107a1 of the back-side cuff 74, which is taken along line XXII-XXII in FIG. 21. As illustrated in FIGS. 21 and 22, the blood pressure measurement device 1 includes two cover members 110, in addition to the components of the second embodiment.

The edge of each of the second wing portions 107a1 is constituted like an arc having a diameter smaller than the inner diameter of the cutout portion 5h at the edge of the back surface 5g of the third wing portion 5f The second wing portions 107a1 are joined by bonding to a region of the wrist 200-side back surface 5g of the third wing portions 5f except for the cutout portion 5g.

The cover member 110 sandwiches the second wing portions 107a1 between the cover member 110 and the third wing portion 5f. A surface shape of the cover member 110 is constituted in a surface shape in which a part of the outer edge is formed in an arc-like shape, whereas the remaining portion is shaped like a straight line. The cover member 110 makes the edge formed in an arc-like shape face the arc-like edge of the lid portion 5e. The arc-like edge of the cover member 110 has the same diameter as that of the arc-like edge of the lid portion 5e.

The cover member 110 includes an edge 111 constituted at the arc-like edge. The cover member 110 is formed such that the edge 111 is higher compared to the other portions of the cover member 110. The edge 111 is disposed at the cutout portion 5h of the lid portion 5e. The height as used herein is the height from the wrist 200-side back surface 112 of the cover member 110.

In addition, the height of the edge 111 has a dimension that allows the two bonding layers 108 and the second wing portions 107a1 to be sandwiched between the region of the cover member 110 except for the edge 111 and the region of the third wing portions 5f of the lid portion 5e except for the cutout portion 5h when the tip of the edge 111 is joined to the cutout portion 5h using the bonding layer 108.

In the cover member 110 configured as described above, the edge 111 is joined to the cutout portion 5h of the lid portion 5e by bonding with the bonding layer 108. In addition, the surface region of the cover member 110 on the lid portion 5e side other than the edge 111 are joined by being bonded to the second wing portions 107a1 by the bonding layer 108.

The blood pressure measurement device 1 according to the third embodiment configured as described above produces effects similar to the effects of the second embodiment. Furthermore, the second wing portions 107a1 are bonded to the cover member 110 and the curler 5 and sandwiched between the cover member 110 and the curler 5, thus enabling a further increase in the joining strength between the back-side cuff 74 and the curler 5.

Note that the present invention is not limited to the embodiments described above. For example, the timings when the first on-off valve 16A and the second on-off valve 16B are opened and closed during blood pressure measurement are not limited to the timings in the examples described above, and can be set as appropriate. Additionally, although the example has been described in which the blood pressure measurement device 1 performs blood pressure measurement by calculating the blood pressure with the pressure measured during the process of pressurizing the palm-side cuff 71, no such limitation is intended and the blood pressure may be calculated during the depressurization process or during both the pressurization process and the depressurization process.

In addition, in the example described above, the configuration has been described in which the air bag 81 is formed by each of the sheet members 86, but no such limitation is intended, and for example, the air bag 81 may further include any other configuration in order to manage deformation and inflation of the palm-side cuff 71, for example.

Additionally, in the examples described above, the configuration is described in which the back plate 72 includes the plurality of grooves 72a, but no such limitation is intended. For example, for management of the likelihood of deformation and the like, the number, the depth, and the like of the plurality of grooves 72a may be set as appropriate, and the back plate 72 may be configured to include a member that suppresses deformation.

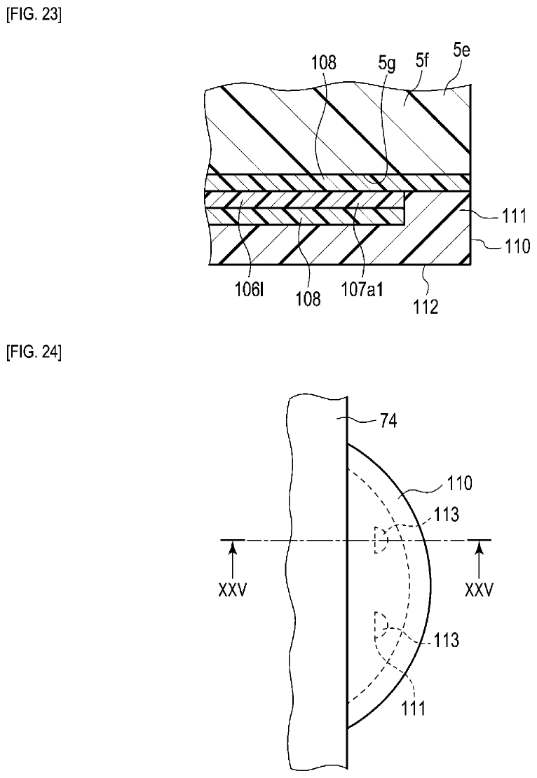

Furthermore, in the third embodiment described above, the configuration has been described in which the lid portion 5e includes the cutout portion 5h. However, no such limitation is intended. For example, as in a modified example illustrated in FIG. 23, a configuration may be provided in which the lid portion 5e does not include the cutout portion 5h.

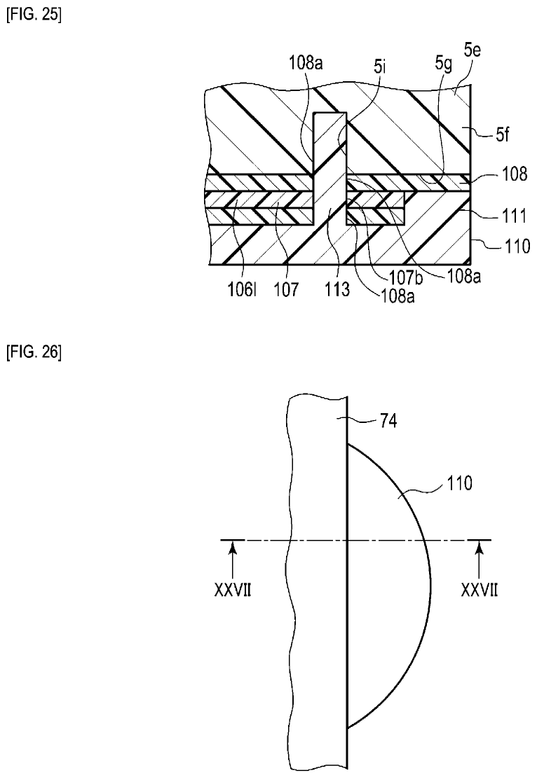

Additionally, in the third embodiment described above, the configuration has been described in which the cover member 110 is joined to the lid portion 5e by bonding with the bonding layer 108. However, no such limitation is intended. For example, as in a modified example illustrated in FIGS. 24 and 25, the cover member 110 may be configured such that a part of the cover member 110 is fixed by fitting to the lid portion 5e. FIG. 24 is a bottom view of the vicinity of one of the two second wing portions 107a1 as viewed from the writ 200 side. FIG. 25 is a cross-sectional view illustrating a configuration of the vicinity of one of the two second wing portions 107a1 of the back-side cuff 74, which is taken along line XXV-XXV in FIG. 24.

As a specific example, as illustrated in FIGS. 24 and 25, holes 5i are formed in the wrist 200-side back surface 5g of the third wing portions 5f of the lid portion 5e. A plurality of, for example, two holes 5i are formed as an example. The two holes 5i are disposed, for example, aligning in the longitudinal direction of the curler 5. The holes 5i are constituted, for example, in a semi-circular shape.

Holes 107b and holes 108a are formed at portions of the second wing portions 107a1 and the bonding layer 108 which portions face the respective holes 5i.

Protruding portions 113 are formed at portions of the cover member 110 facing the holes 107b in the second wing portions 107a1. The protruding portions 113 are inserted through the holes 107b and 108a. Furthermore, a tip-side portion of each of the protruding portion 113 having passed through the hole 108a fits into the hole 5i. The protruding portion 113 is constituted in a columnar shape in which, for example, a cross section orthogonal to the axial direction is formed in a semi-circular shape fitted into the hole 5i. A portion of the protruding portion 113 is disposed in the holes 107 b and 108a. Additionally, the portion of the protruding portion 113 having passed through the hole 108a fits into the hole 5i.

Thus, the protruding portions 113 fits into the holes 5i to mechanically fix the cover member 110 to the curler 5A, enabling an increase in the fixing strength between the cover and the curler 5. Note that in the modified example illustrated in FIGS. 24 and 25, the arc-like edge of the back surface 5g of the lid portion 5e is configured not to include the cutout portion 5h as an example.

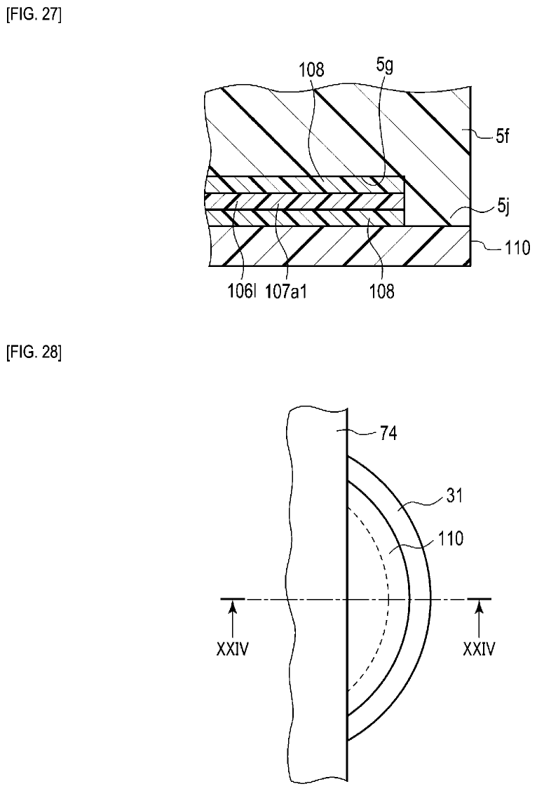

Furthermore, in the third embodiment described above, the configuration has been described in which the cover member 110 is joined to the lid portion 5e by being bonded using the bonding layer 108. However, no such limitation is intended. For example, as in a modified example illustrated in FIGS. 26 and 27, the cover member 110 may be joined to the lid portion 5e by welding. FIG. 26 is a bottom view of the vicinity of one of the two second wing portions 107a1 of the back-side cuff 74 as viewed from the wrist 200 side. FIG. 27 is a cross-sectional view illustrating a configuration of the vicinity of one of the two second wing portions 107a1 of the back-side cuff 74, which is taken along line XXVII-XXVII in FIG. 26.

As illustrated in FIGS. 26 and 27, an arc-like edge of the back surface 5g of the third wing portion 5f is constituted in a protruding portion 5j that protrudes toward the cover member 110 side compared to the other. The height of the protruding portion 5j has a dimension that allows the second wing portion 107a1 and the two bonding layers 108 to be sandwiched between a region of the back surface 5g other than the protruding portions 5j and the cover member 110. The edge 111 of the cover member 110 is joined by welding to the edge of the third wing portion 5f. The means for welding is thermocompression bonding, as an example.

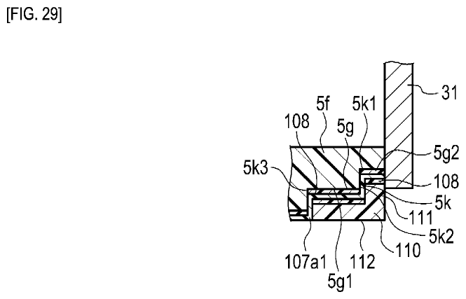

Furthermore, in the third embodiment described above, a configuration has been described in which the cover member 110 is joined by being bonded to the lid portion 5e by the bonding layer 108, no such limitation is intended. For example, as in the modified example illustrated in FIGS. 28 and 29, the cover member 110 may be fixed to the outer case 31 by fitting into the outer case 31 in addition to joining with the bonding layer 108.

FIG. 28 is a bottom view of the vicinity of one of the two second wing portions 107a1 of the back-side cuff 74 as viewed from the wrist 200 side. FIG. 29 is a cross-sectional view illustrating a configuration of the vicinity of one of two second wing portions 107a1 of the back-side cuff 74, which is taken along line XXIV-XXIV in FIG. 28.

As a specific example, the back surface 5g of the third wing portion 5f is constituted be in a shape that is one step lower than the back surface at an inner side in the width direction of the third wing portion 5f of the lid portion 5e. Specifically, as illustrated in FIG. 29, the back surface 5g is positioned above compared to the inner region of the back surface of the lid portion 5e in the width direction of the third wing portion 5f.

Furthermore, a side surface 5k of the third wing portion 5f constituted on the arc surface of the lid portion 5e includes a plurality of arc surfaces having different diameters. As a further specific example, the side surface 5k includes a first arc surface portion 5k1, a second arc surface portion 5k2, and a third arc surface portion 5k3.

The first arc surface portion 5k1 is a portion of the side surface 5k constituted to have the largest diameter. The second arc surface portion 5k2 is constituted as an arc surface having a center of curvature coaxial with the center of curvature of the first arc surface portion 5k1. The second arc surface portion 5k2 is constituted as an arc surface having a smaller diameter than the first arc surface portion 5k1. The third arced surface portion 5k3 is constituted as an arc surface having a center of curvature coaxial with the center of curvature of the first arc surface portion 5k1. The third arced surface portion 5k3 is constituted as an arc surface having a smaller diameter than the second arc surface portion 5k2.

In addition, because the side surface 5k includes the arc surface portion 5k1, 5k2, and 5k3, the back surface 5g of the third wing portion 5f includes a first back surface portion 5g1 disposed in the center, and a second back surface portion 5g2 disposed on the outer side of the first back surface portion 5g1.

The second wing portions 107a1 are joined to the first back surface portion 5g1 and the second back surface portion 5g2 by bonding with the bonding layer 108.

As a specific example, the cover member 110 is joined to a region of the second wing portion 107a1 joined to the second back surface portion 5g2 by bonding the edge 111 to the region using the bonding layer 108. The region of the cover member 110 except for the edge 111 is joined, by bonding with the bonding layer 108, to a region of the second wing portion 107a joined to the first back surface portion 5b1.

Apart of the cover member 110 and the lid portion 5e are fitted inside the outer case 31.

Thus, a part of the cover member 110 and the lid portion 5e fit inside the outer case 31, enabling an increase in the fixing strength between the curler 5A and the case 11, and the fixing strength between the cover member 110 and the case 11 can be increased. Furthermore, the junction portion between the cover member 110 and the lid portion 5e is covered by the outer case 31, thus allowing improvement of sealability of the junction portion between the cover member 110 and the lid portion 5e. The junction portion as used herein is a portion including a line end surface of the edge 111, the bonding layer 108, and the second back surface portion 5g2.

Additionally, in the third embodiment described above, the configuration has been described in which the second wing portions 107a is joined to the lid portion 5e using the bonding layer 108. However, no such limitation is intended. For example, the second wing portion 107a may be joined to the lid portion 5e using a joining member. The joining member is a member that mechanically joins two members, and examples of the joining member include protrusions for swaging, rivets, sewing threads, and the like.

In addition, the above-described modified examples, illustrated in FIGS. 21 to 29, are configured such that the curler 5A includes the lid portion 5e. However, no such limitation is intended. As in the first embodiment, the modified examples illustrated in FIGS. 21 to 29 may be configured to include the case 11 with the back lid 35 and the curler 5 with the first facing portion 5a.

In addition, in the above-described first to third embodiments and the modified examples of the third embodiment illustrated in FIGS. 21 to 29, as an example, the configuration has been described in which the curler 5 includes the first facing portion 5a that is aligned at an end portion of the outer case 31 in the thickness direction and that is larger than the other portions of the curler 5 in the width direction, and the eighteenth sheet member 106l includes the second facing portion 107 that faces the first facing portion 5a of the curler 5 and that is larger in the width direction than the other portions of the eighteenth sheet member 106l. However, no such limitation is intended. In other examples, the first facing portion 5a of the curler 5 may be configured to have the same width in the width direction as the other portions. Even in this configuration, the junction margin is increased by the second facing portion 107 of the eighteenth sheet member 106l, and thus joining strength between the back-side cuff 74 and the curler 5 can be increased.

In other words, the embodiments described above are merely examples of the present invention in all respects. Of course, various modifications and variations can be made without departing from the scope of the present invention. Thus, specific configurations in accordance with an embodiment may be adopted as appropriate at the time of carrying out the present invention.

### REFERENCE SIGNS LIST

1 Blood pressure measurement device
3 Device main body
4 Belt
5, 5A Curler
5a First facing portion
5a1 First wing portion
5e Lid portion
5f Third wing portion
5g Back surface
5g1 First back surface portion
5g2 Second back surface portion
5i Hole
5j Protruding portion
6 Cuff structure
7 Fluid circuit
7a First flow path
7b Second flow path
7c Third flow path
11 Case
12 Display unit
13 Operation unit
14 Pump
15 Flow path unit
16 On-off valve
16A First on-off valve
16B Second on-off valve
17 Pressure sensor
17A First pressure sensor
17B Second pressure sensor
18 Power supply unit
19 Vibration motor
20 Control substrate
31 Outer case
31a Lug
31b Spring rod
32 Windshield
33 Base
35 Back lid
35a Screw
41 Button
42 Sensor
43 Touch panel
51 Substrate
52 Acceleration sensor
53 Communication unit
54 Storage unit
55 Control unit
61 First belt
61a First hole portion
61b Second hole portion
61c Buckle
61d Frame body
61e Prong
62 Second belt
62a Small hole
62b Third hole portion
71 Palm-side cuff (cuff)
71A Pressing cuff
72 Back plate
72a Groove
73 Sensing cuff
74 Back-side cuff (cuff)
75 Flat plate
76 Bag-like cover body
81 Air bag (bag-like structure)
81a Welded portion
82 Insertion hole
86 Sheet member
86a First sheet member
86b Second sheet member
86b1 Opening
86c Third sheet member
86c1 Opening
86d Fourth sheet member
91 Air bag (bag-like structure)
92 Tube
93 Connection unit
96 Sheet member
96a Fifth sheet member
96b Sixth sheet member
101 Air bag (bag-like structure)
101a Welded portion
102 Tube
103 Connection portion
106 Sheet member
106a Seventh sheet member
106b Eighth sheet member
106b1 Opening
106c Ninth sheet member
106c1 Opening
106d Tenth sheet member
106d1 Opening
106e Eleventh sheet member
106e1 Opening
106f Twelfth sheet member
106f1 Opening
106g Thirteenth sheet member
106g1 Opening
106h Fourteenth sheet member
106h1 Opening
106i Fifteenth sheet member
106i1 Opening
106j Sixteenth sheet member
106j1 Opening
106k Seventeenth sheet member
106k1 Opening
106l Eighteenth sheet member
107, 107A Second facing portion
107a Second wing portion
107a1 Second wing portion
107b Hole
108 Bonding layer
110 Cover member
111 Edge
113 Protruding portion
200 Wrist
210 Artery

1. A blood pressure measurement device comprising:
a case including an outer case having a tubular shape;
a curler curving in such a manner as to follow along a circumferential direction of a portion of a living body where the blood pressure measurement device is attached, the curler including a first facing portion aligned at one end of the outer case in a thickness direction; and
a cuff formed of two sheet members formed of a resin material, the cuff being configured to be inflated with a fluid, one of the sheet members that is disposed on the curler side including a second facing portion facing the first facing portion, and the second facing portion being larger than other portions of the cuff in a width direction.

2. The blood pressure measurement device according to claim 1, wherein the first facing portion is constituted in a shape being larger than other portions of the curler in a width direction.

3. The blood pressure measurement device according to claim 2, further comprising a cover member sandwiching a portion of the second facing portion between the cover member and the first facing portion, the portion protruding beyond the other portions in the width direction.

4. The blood pressure measurement device according to claim 3, wherein
the cover member includes a protruding portion,
the second facing portion includes a hole where a part of the protruding portion is disposed, and
the first facing portion includes a fitting portion where a part of the protruding portion fits.

5. The blood pressure measurement device according to claim 1, wherein
the case includes a back lid covering the one end of the outer case, and
the first facing portion is connected to the back lid.

6. The blood pressure measurement device according to claim 2, wherein the first facing portion covers the one end of the outer case.
